SEQUENCE LISTING (6 sequences)

SEQ ID NO: 1 - DNA, 23 bp, Artificial Sequence
  IL1b FWD primer
tgtgctctgcttgtgaggtgctg

SEQ ID NO: 2 - DNA, 22 bp, Artificial Sequence
  IL1b REV primer
ccctgcagctggagagtgtgga

SEQ ID NO: 3 - DNA, 20 bp, Artificial Sequence
  Hprt1 FWD primer
cagtcccagcgtcgtgatta

SEQ ID NO: 4 - DNA, 20 bp, Artificial Sequence
  Hprt1 REV primer
tggcctcccatctccttcat

SEQ ID NO: 5 - DNA, 20 bp, Artificial Sequence
  18S FWD primer
gtaacccgttgaaccccatt

SEQ ID NO: 6 - DNA, 20 bp, Artificial Sequence
  18S REV primer
ccatccaatcggtagtagcg

(19) United States
(12) Patent Application Publication
(10) Pub. No.: US 2022/0378907 A1
(43) Pub. Date: Dec. 1, 2022

(54) METHODS FOR TREATING DISEASES

(71) Applicant: CHILDREN'S HOSPITAL MEDICAL CENTER, Cincinnati, OH (US)

(72) Inventors: Chandrashekhar PASARE, Cincinnati, OH (US); Aakanksha JAIN, Boston, MA (US)

(73) Assignee: CHILDREN'S HOSPITAL MEDICAL CENTER, Cincinnati, OH (US)

(21) Appl. No.: 17/755,787

(22) PCT Filed: Nov. 12, 2020
(86) PCT No.: PCT/US2020/060090

§ 371 Date: May 7, 2022

Related U.S. Application Data
(60) Provisional application No. 62/934,805, filed on Nov. 13, 2019.
(60) Provisional application No. 62/934,793, filed on Nov. 13, 2019.

(51) Int. Cl. A61K 39/395; A61K 38/17; A61K 31/454; A61K 31/522; A61K 31/137; A61K 31/135; A61K 31/48; A61K 31/12; A61K 31/353; A61K 31/05; A61K 31/197; A61P 37/06

(52) U.S. Cl. CPC A61K 39/3955; A61K 38/1793; A61K 31/454; A61K 31/522; A61K 31/137; A61K 31/135; A61K 31/48; A61K 31/12; A61K 31/353; A61K 31/05; A61K 31/197; A61P 37/06

(57) ABSTRACT

Some embodiments of the invention include methods for treating an animal for a disease comprising one or more administrations of one or more compositions comprising (a) a TNF signaling inhibitor, (b) a CD40 inhibitor, a FAS signaling inhibitor, or both, and (c) optionally, a caspase 8 inhibitor. Other embodiments include methods for treating the disease comprising one or h more administrations of one or more compositions comprising (a) the TNF signaling inhibitor and (b) the CD40 inhibitor. Certain embodiments include methods for treating the disease comprising one or more administrations of one or more compositions comprising (a) the TNF signaling inhibitor, (b) the FAS signaling inhibitor, and (c) optionally, the caspase 8 inhibitor. Still other embodiments include methods for treating a human for autoimmune disease, T cell mediated autoimmune disease, IL-1β mediated autoimmune disease, or cytokine release syndrome. Additional embodiments of the invention are also discussed herein.

### CROSS-REFERENCE TO RELATED APPLICATIONS

This application claims the benefit of U.S. Provisional Application No. 62/934,793, filed Nov. 13, 2019, entitled “Methods for Treating Diseases” which is herein incorporated by reference in its entirety.

This application also claims the benefit of U.S. Provisional Application No. 62/934,805, filed Nov. 13, 2019, entitled “Methods for Treating Diseases” which is herein incorporated by reference in its entirety.

### GOVERNMENT RIGHTS

This invention was made with government support under AI123176 awarded by the National Institutes of Health. The government has certain rights in the invention.

### BACKGROUND

Diseases continue to plague animals (e.g., humans). Indeed, many diseases, including but not limited to autoimmune diseases, do not have cures or treatments that are adequate.

Some embodiments of the invention include methods for treating an animal for a disease comprising one or more administrations of one or more compositions comprising (a) a TNF signaling inhibitor, (b) a CD40 inhibitor, a FAS signaling inhibitor, or both, and (c) optionally, a caspase 8 inhibitor. Other embodiments include methods for treating the disease comprising one or more administrations of one or more compositions comprising (a) the TNF signaling inhibitor and (b) the CD40 inhibitor. Certain embodiments include methods for treating the disease comprising one or more administrations of one or more compositions comprising (a) the TNF signaling inhibitor, (b) the FAS signaling inhibitor, and (c) optionally, the caspase 8 inhibitor. Still other embodiments include methods for treating a human for autoimmune disease, T cell mediated autoimmune disease, IL-1β mediated autoimmune disease, or cytokine release syndrome. Additional embodiments of the invention are also discussed herein.

### SUMMARY

Some embodiments of the invention include a method for treating a disease comprising one or more administrations of one or more compositions comprising (a) a TNF signaling inhibitor, (b) a CD40 inhibitor, a FAS signaling inhibitor, or both, and (c) optionally a caspase 8 inhibitor. In other embodiments, the method for treating the disease comprises one or more administrations of one or more compositions comprising (a) the TNF signaling inhibitor and (b) the CD40 inhibitor. In still other embodiments, the method for treating the disease comprises one or more administrations of one or more compositions comprising (a) the TNF signaling inhibitor, (b) the FAS signaling inhibitor, and (c) optionally the caspase 8 inhibitor. In yet other embodiments, the TNF signaling inhibitor is a TNFR inhibitor, TNFR1 inhibitor, TNFR2 inhibitor, a TNF inhibitor, or a TNFα inhibitor. In certain embodiments, the TNF signaling inhibitor is a TNFR inhibitor and the TNFR inhibitor is infliximab (Remicade), adalimumab (Humira), certolizumab pegol (Cimzia), golimumab (Simponi), or etanercept (Enbrel). In some embodiments, TNF signaling inhibitor is a TNF inhibitor and the TNF inhibitor is infliximab (Remicade) or biosimilars thereof, adalimumab (Humira) or biosimilars thereof, certolizumab or biosimilars thereof, certolizumab pegol (Cimzia) or biosimilars thereof, golimumab (Simponi) or biosimilars thereof, etanercept (Enbrel) or biosimilars thereof, Remsima, Thalidomide (Immunoprin) or derivatives thereof, lenalidomide (Revlimid), pomalidomide (Pomalyst, Imnovid), xanthine derivatives, pentoxifylline, bupropion, 5-HT2A agonist, (R)-DOI, TCB-2, LSD, LA-SS-Az, curcumin, catechins, or Cannabidiol. In other embodiments, the CD40 inhibitor is an anti-CD40L antibody, BI 655064, CFZ533, BG9588, or KPL-404. In yet other embodiments, the FAS signaling inhibitor is an anti-FasL Ab. In certain embodiments, at least one of the one or more compositions comprises the caspase 8 inhibitor and the caspase 8 inhibitor is Emricasan. In still other embodiments, the amount of the TNFR inhibitor is from about 0.0001% (by weight total composition) to about 99%. In certain embodiments, the amount of the TNF inhibitor is from about 0.0001% (by weight total composition) to about 99%. In some embodiments, the amount of the CD40 inhibitor is from about 0.0001% (by weight total composition) to about 99%. In other embodiments, the amount of the FAS inhibitor is from about 0.0001% (by weight total composition) to about 99%. In yet other embodiments, the amount of the caspase 8 inhibitor is from about 0.0001% (by weight total composition) to about 99%. In some embodiments, at least one of the one or more compositions further comprises a formulary ingredient. In other embodiments, at least one of the one or more compositions is a pharmaceutical composition.

In still other embodiments, at least one of the one or more administrations comprises a parenteral administration, a mucosal administration, an intravenous administration, a depot injection, a subcutaneous administration, a topical administration, an intradermal administration, an oral administration, a sublingual administration, an intratracheal administration, an intranasal administration, an intramuscular administration, an aerosol administration, a nebulizer administration, a pressurized metered-dose inhaler (pMDI) administration, an inhaler administration, or a dry powder inhaler (DPI) administration. In yet other embodiments, at least one of the one or more administrations comprises an intravenous administration, a depot injection, a subcutaneous administration, a topical administration, an oral administration, a sublingual administration, or an intramuscular administration. In certain embodiments, if there is more than one administration (a) at least one composition used for at least one administration is different from the composition of at least one other administration, (b) the TNF signaling inhibitor is in one composition and the CD40 signaling inhibitor is in another composition, or (c) the TNF signaling inhibitor and the CD40 signaling inhibitor are in same composition. In some embodiments, if there is more than one administration (a) at least one composition used for at least one administration is different from the composition of at least one other administration, (b) the TNF signaling inhibitor is in one composition and the FAS signaling inhibitor is in another composition, or (c) the TNF signaling inhibitor and the FAS signaling inhibitor are in same composition. In other embodiments, the TNF signaling inhibitor is a TNFR inhibitor and the TNFR inhibitor of at least one of the one or more compositions is administered to the animal in an amount of from about 0.005 mg/kg animal body weight to about 100 mg/kg animal body weight. In still other embodiments, the TNF signaling inhibitor is a TNF inhibitor and the TNF inhibitor of at least one of the one or more compositions is administered to the animal in an amount of from about 0.005 mg/kg animal body weight to about 100 mg/kg animal body weight. In yet other embodiments, the CD40 inhibitor of at least one of the one or more compositions is administered to the animal in an amount of from about 0.005 mg/kg animal body weight to about 100 mg/kg animal body weight. In certain embodiments, the FAS inhibitor of at least one of the one or more compositions is administered to the animal in an amount of from about 0.005 mg/kg animal body weight to about 100 mg/kg animal body weight. In some embodiments, the caspase 8 inhibitor of at least one of the one or more compositions is administered to the animal in an amount of from about 0.005 mg/kg animal body weight to about 100 mg/kg animal body weight. In other embodiments, the animal is a human, a rodent, or a primate. In still other embodiments, the animal is in need of treatment of the disease. In yet other embodiments, the method is for treating an autoimmune disease, a T cell mediated autoimmune disease, an IL-1β mediated autoimmune disease, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis, uveitis, or cytokine release syndrome. In certain embodiments, the method is for treating a T cell mediated autoimmune disease, type 1 diabetes, rheumatoid arthritis, multiple sclerosis, or Celiac disease. In some embodiments, the method is for treating an IL-1β mediated autoimmune disease, type 1 diabetes, pericarditis, rheumatoid arthritis, or psoriasis. In other embodiments, the method is for preventing cytokine release syndrome.

Some embodiments of the invention include a method for treating a disease comprising one or more administrations of one or more compositions comprising (a) a TNF signaling inhibitor and (b) a CD40 inhibitor, wherein the disease is an autoimmune disease, a T cell mediated autoimmune disease, an IL-1β mediated autoimmune disease, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis, uveitis, or cytokine release syndrome.

Some embodiments of the invention include a method for treating a disease comprising one or more administrations of one or more compositions comprising (a) a TNF signaling inhibitor, (b) a FAS signaling inhibitor, and (c) optionally a caspase 8 inhibitor, wherein the disease is an autoimmune disease, a T cell mediated autoimmune disease, an IL-1β mediated autoimmune disease, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis, uveitis, or cytokine release syndrome.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: Cognate interaction between BMDCs and effector CD4 T cells leads to inflammasome independent production of bioactive IL-1β by BMDCs. (a) IL-1β, as quantified by ELISA, in the supernatants of wild-type (WT) effector CD4⁺ cells (T_{H}0 cells) co-cultured with WT BMDCs in the presence or absence of CD3 Ab for 18 h. Error bars indicate SEM for n=3 independent experiments. (b) Production of IL-1b measure by ELISA in supernatants of effector CD4⁺ T cells polarized to T_{H}1, T_{H}2 and T_{H}17 lineages cultured with WT BMDCs in the presence of CD3 Ab for 18 h or left untreated. Error bars indicate SEM for n=3 independent experiments. (c-d) IL-1β in the supernatants following stimulation of T_{H}17-polarized OT-II T cells for 18 h with fixed (100 μM) (c) or titrating (d) concentrations of OT-II₃₂₃₋₃₃₉ peptide presented by WT BMDCs. NT, no T cells in culture. Error bars indicate SEM from n=2 technical replicates. Data are representative of 3 independent experiments. (e) IL-1β measured by ELISA after 18 h of culture of effector WT or Il1b^{-/-} CD4⁺ T_{H}0 cells with WT or Il1b^{-/-} BMDCs in the presence of CD3 Ab. Error bars indicate SEM from two (n=2) independent experiments. (f) qPCR of Il1b mRNA in lysates of WT BMDCs co-cultured with Il1b^{-/-} T_{H}0 cells and treated with CD3 Ab for 3 h. Data are normalized to 18s rRNA. Error bars indicate SEM from n=2 technical replicates. Data are representative of three independent experiments. (g) Expression of intracellular pro-IL-1β measured by flow cytometry in WT live, CD90⁻CD11c⁺ BMDCs stimulated with LPS (100 ng/mL) or cultured with T_{H}0 cells in the presence of CD3 Ab for 6 h. Data are representative of three independent experiments. (h) Immunoblot analysis of pro-IL-1β (p35) or cleaved IL-1β (p17) in the cell lysates or the supernatants, respectively, of WT BMDCs co-cultured with WT T_{H}0 cells treated with CD3 Ab for 18 h. Data are representative of three independent experiments. (a, b) Statistical analysis was performed by paired, one-tailed Student's t-test. Individual p values: **p<0.01, ***p<0.001, ****p<0.0001.

Representative gating strategy for flow cytometric analysis (i) Gating strategy for flow cytometric analysis of intracellular pro-IL-1β in CD11c⁺ DCs following BMDC-T cell co-culture. (j) Gating strategy for flow cytometric analysis of CD11b⁺ monocytes and neutrophils frequency in various tissues.

FIG. 2: T cell derived TNFα for induction of pro-IL-1b in BMDCs. (a) IL-1β, as quantified by ELISA, in the supernatants of WT T_{H}0 cells co-cultured with WT or Tlr2/4^{-/-} BMDCs in the presence of CD3 Ab for 6 h. Error bars indicate SEM for n=4 independent experiments. (b) IL-1β measured by ELISA following 6 h of culture with WT T_{H}0 cells and WT BMDCs in the presence of CD3 Ab and neutralizing TNF Ab (20 μg/mL), FasL Ab (10 μg/mL), or CD40L Ab (20 μg/mL). Error bars indicate SEM for n=3 independent experiments. (c) Immunoblot analysis of pro-IL-1β (p35) in the lysates of WT BMDCs stimulated with LPS (100 ng/mL) or cultured with T_{H}0 cells in the presence of CD3 Ab and neutralizing antibodies. Data are representative of two independent experiments. (d) Expression of intracellular pro-IL-1β measured by flow cytometry in WT live, CD90⁻CD11c⁺ BMDCs cultured with T_{H}0 cells in the presence of CD3 Ab and neutralizing TNF Ab (20 μg/mL) for 6 h. Flow plots are representative of four independent experiments. Error bars indicate SEM for n=4 independent experiments. (e) Expression of intracellular TNF measured by flow cytometry in WT T_{H}0 cells (left; live, CD11c⁻CD90.2⁺) and WT BMDCs (right; live, CD90.2⁻CD11c⁺) that were co-cultured for 3 h in the presence of CD3 Ab and brefeldin A. Data are representative of two independent experiments. (f) Expression of intracellular TNF measured by flow cytometry in effector CD4⁺ T cells (live, CD11c⁻CD90.2⁺) polarized to TH1, TH2 and T_{H}17 lineages cultured with WT BMDCs in the presence of CD3 Ab and brefeldin A for 3 h. Cells were considered to be transcription factor positive based on isotype control antibody staining. Data are representative of three independent experiments. (g) Immunoblot analysis of pro-IL-1β (p35) in the lysates of WT or Tnf^{-/-} T_{H}0 cells cultured WT BMDCs in the presence or absence of CD3 Ab for 6 h. Data are representative of two independent experiments. (h) IL-1β was quantified by ELISA in the supernatants of WT T_{H}0 cells co-cultured with WT or Tnfrsf1a^{-/-}Tnfrsf1b^{-/-} BMDCs in the presence of CD3 Ab for 6 h. Error bars indicate SEM for n=4 independent experiments. (a, b, d, h) Statistical analysis was performed by paired, one-tailed Student's t-test. *p<0.05, **p<0.01, ***p<0.001, n.s.=not significant.

Induction of pro-IL-1β in BMDCs is independent of TLR activation but dependent on TNF. (i and j) Expression of intracellular pro-IL-1β measured by flow cytometry in WT, Tlr2⁻/−Tlr4^{-/-}, or Myd88^{-/-} BMDCs (live, CD90-CD11c⁺) cultured with T_{H}0 cells in the presence of CD3 Ab for 6 h. Fold change indicates proportion of pro-IL-1β⁺ BMDCs, compared to PBS controls. Error bars indicate SEM three (n=3) independent experiments. (k) Expression of intracellular pro-IL-1β measured by flow cytometry in WT BMDCs (live, CD11c⁺) stimulated in vitro with recombinant TNFα(20 ng/mL) for 6 h. Data are representative of two independent experiments. (1) TNFα, as quantified by ELISA, in the supernatants of WT or Tnf^{-/-} T_{H}0 cells cultured with WT or Tnf BMDCs in the presence of CD3 Ab for 6 h. Error bars indicate SEM from n=4 independent experiments. (m) IL-1β was quantified by ELISA in the supernatants of WT T_{H}0 cells cultured with BMDCs of the indicated genotypes in the presence of CD3 Ab and neutralizing CD40L Ab (10 μg/mL) for 6 h. Error bars indicate SEM from n=2 technical replicates. Data are representative of two independent experiments. (i, g, l) Statistical analysis was performed by paired, two-tailed Student's t-test. *p<0.05.

FIG. 3: T cell instructed IL-1β production is independent of Caspase-1. (a) Immunoblot analysis of pro-caspl (p45) and cleaved caspl (p20) in the lysates of WT BMDCs stimulated with LPS+ATP or cultured with WT T_{H}0 cells in the presence of CD3 Ab for 18 h. Data are representative of three independent experiments. (b) Immunoblot analysis of cleaved IL-1β (p17) or (c) IL-1β, as quantified by ELISA, in the supernatant of WT or Casp1^{-/-} BMDCs cultured with WT T_{H}0 cells in the presence of CD3 Ab for 18 h. (b) Data are representative of three independent experiments. (c) Error bars indicate SEM for n=3 independent experiments. Statistical analysis was performed by paired, one-tailed Student's t-test. n.s.=not significant. (d) IL-1β measured by ELISA following 18 h of culture with WT T_{H}0 cells and WT, Casp1/11^{-/-}, or Pycard^{-/-} BMDCs in the presence of CD3 Ab. Data are representative of two independent experiments. Error bars indicate SEM for n=2 technical replicates.

T cell-induced IL-1β production is partially impaired in gasdermin-D deficient BMDCs (e) IL-1b was quantified by ELISA in the supernatants of WT TH0 cells cultured with Il1b^{-/-} BMDCs transduced with retrovirus expressing WT or D117A pro-IL-1β in the presence of CD3 Ab for 18 h. Error bars indicate SEM from n=2 technical replicates. Data are representative of two independent experiments. (f) IL-1β was quantified by ELISA in the supernatants of WT TH0 cells cultured with WT or Gsdmd^{-/-} BMDCs in the presence of CD3 Ab for 12 h. Error bars indicate SEM from n=6 independent experiments. Statistical analysis was performed by paired, two-tailed Student's t-test. **p<0.01.

FIG. 4: Fas-FasL interaction between effector CD4 T cells and BMDCs leads to caspase-8 dependent cleavage of pro-IL-1β. (a) IL-1β, as quantified by ELISA, or (b) detected by immunoblot analysis of cleaved IL-1β (p17) in the supernatants of WT T_{H}0 cells co-cultured with WT or lpr BMDCs in the presence of CD3 Ab and neutralizing FasL Ab (10 μg/mL) for 18 h. (a) Error bars indicate SEM for n=3 independent experiments. (b) Data are representative of two independent experiments. (c) Immunoblot analysis of cleaved casp-8 (p18) in the lysates of WT BMDCs cultured with WT T_{H}0 cells in the presence of CD3 Ab and neutralizing FasL Ab (10 μg/mL) or TNF Ab (20 μg/mL) for 12 h. Data are representative of two independent experiments. (d) Immunoblot analysis of pro-IL-1β (p35) or cleaved IL-1β (p17) in the cell lysates or the supernatants, respectively, of WT BMDCs co-cultured with WT T_{H}0 cells in the presence of CD3 Ab and IETD (10 μM) for 18 h. Data are representative of two independent experiments. (e) IL-1β was quantified by ELISA in the supernatants of WT T_{H}0 cells co-cultured with WT, Rip3^{-/-}, or Rip3^{-/-}Casp8^{-/-} BMDCs in the presence of CD3 Ab for 18 h. Error bars indicate SEM for n=3 independent experiments. Statistical analysis was performed by paired, one-tailed Student's t-test. *p<0.05.

T cells induce Fas-caspase-8 dependent death of interacting BMDCs. (f) Expression of surface FasL measured by flow cytometry in in vitro polarized TH1, TH2 and TH₁₇ cells (live, CD90.2⁺) cultured with WT BMDCs in the presence of CD3 Ab for 1 h. Data are representative of two independent experiments. (g and h) Cell death as assayed by Zombie Yellow viability dye was measured by flow cytometry in WT, Ripk3^{-/-}, or Ripk3^{-/-}Casp8^{-/-} live, CD90.2⁺CD11c⁺ BMDCs cultured with WT TH0 cells in the presence or absence of CD3 Ab and neutralizing TNF Ab (20 μg/mL) or FasL Ab (10 μg/mL) for 6 h. Data are representative of two independent experiments.

FIG. 5: Diverse myeloid cells populations utilize TNFR-Fas pathway for T cell induced IL-1β production. IL-1β was quantified by ELISA in the supernatants of WT T_{H}0 cells co-cultured with (a) CD11b⁺ BMDCs, CD11b⁺CD11c⁺MHCII^{Hi} BMDCs, CD11b⁺CD11c⁺MHCII^{int} BMDCs, (b) CSF1R⁻ CD11c⁺MHCII^{Hi} GM-DCs, or CSF1R⁺CD11c⁺MHCII^{int} GM-Macs in the presence of CD3 Ab and neutralizing TNF Ab (20 μg/mL) or FasL Ab (10 μg/mL) for 18 h. IL-1β was quantified by ELISA in the supernatants of WT T_{H}0 cells co-cultured with (c) BMDMs, or (d) CD11c⁺ cDCs FACS sorted from WT spleen (left) or CD11c⁺Zbtb46-GFP⁺Ly6C⁻ splenic cDCs FACS sorted from Zbtb46-GFP reporter mice spleen (right) as in (a). Error bars indicate SEM for n=3-4 independent experiments. (e) IL-17A was quantified by ELISA in the supernatants of WT CD44^{hi}CD62L^{lo} effector CD4 T cells re-stimulated with CD3 Ab using WT or Tnfrsf1la^{-/-}Tnfrsf1b^{-/-} CD11c⁺ splenic cDCs or (f) WT CD11c⁺ splenic cDCs in the presence of neutralizing FasL Ab (10 μg/mL) for 48 h. Error bars indicate SEM from n=4 independent experiments. Statistical analysis was performed by paired, one-tailed Student's t-test. *p<0.05, **p<0.01, ***p<0.001, ****p<0.0001.

Gating strategy and post sort purity of various myeloid cell populations. (g and h) Pre-sort and post-sort purity of FACS sorted BMDC subsets: CD11b⁺, CD11b⁺CD11c⁺MHCII^{hi}, or CD11b⁺CD11c⁺MHCII^{int} BMDCs (h) and CD11c⁺MHCII^{int}CSF1R⁺ (GM-Macs) or CD11c⁺MHCII^{hi}CSF1R⁻ (GM-DCs). Data are representative of 4 independent experiments. (i) Post-sort purity of CD11c⁺ splenic DCs showing lack of CSF1R⁺ cell contamination. Data are representative of 4 independent experiments. (j) Pre-sort and post-sort purity of CD11c⁺Zbtb46-GFP⁻Ly6C⁻ splenic cDCs that were FACS sorted from pre-sorted total CD11c⁺ Zbtb46-GFP splenocytes to obtain a pure cDC population. Data are representative of three independent experiments.

FIG. 6: CD4 T cells engage TNFR and Fas signaling pathways to induce IL-1β mediated systemic inflammation. (a) qPCR of Il1b mRNA in lysates of splenocytes collected from WT mice 3-4 h post CD3 Ab injection (50 μg, i.v.). Data are normalized to Hprt1. Error bars indicate SEM from n=3 technical replicates. Data are representative of three independent experiments. (b) Expression of intracellular pro-IL-1β measured by flow cytometry in WT live, CD11c⁺ splenocytes from (a). Data are representative of 4 independent experiments. Statistical analysis was performed by paired one-tailed Student's t-test. *p<0.05. (c) Neutrophil infiltration in the SI-LP or (d) spleen as measured by flow cytometry in WT or Il1b^{-/-} mice 18 h post CD3 Ab injection (20 μg, i.p.) Error bars indicate SEM from n=3-4 independent experiments. (e) Neutrophil infiltration in the spleen as measured by flow cytometry 12 h post OVA₃₂₃₋₃₃₉ peptide injection (50 μg, i.v.) into WT or Il1b^{-/-} mice that previously received (1 d prior) OT-II T_{H}17 cells (5×10⁶, i.v.). Error bars indicate SEM from n=4 independent experiments. (f) qPCR of Il1b mRNA in lysates of splenocytes collected from WT or Tnf^{-/-} mice 3-4 h post CD3 Ab injection (50 μg, i.v.). Data are normalized to Hprt1. Error bars indicate SEM from n=3 technical replicates. Data are representative of two independent experiments. (g) Neutrophil infiltration in the spleen as measured by flow cytometry in WT and Tnf mice 3 h post CD3 Ab injection or (h) Fas^{fl/fl} and Fas^{fl/fl}×CD11c-cre (Fas^{ΔDC}) mice 18 h post CD3 Ab injection (20 μg, i.p.). All Ly6G⁺ cells were previously gated on live, CD11b⁺F480⁻ cells. Error bars indicate SEM from n=4-6 independent experiments. (a, c, d, e, g) Statistical analysis was performed by unpaired, one-tailed Student's t-test. *p<0.05, **p<0.01, ***p<0.001, ****p<0.0001.

Anti-CD3 stimulation of T cells in vivo leads to IL-1β dependent inflammatory cell recruitment dictated by Fas expression on CD11c⁺ cells (i) Expression of intracellular pro-IL-1β measured by flow cytometry in CD11b⁺Ly6C⁻CSF1R⁺ macrophages, CD11b⁺Ly6G⁻Ly6C^{int} monocytes, CD11b⁺Ly6C^{hi} inflammatory monocytes, and CD11b⁺Ly6G⁺ granulocytes quantified from the spleens of WT mice 3-4 h post CD3 Ab injection (50 μg, i.v.). n=3 independent experiments are quantified in the inset. Statistical analysis was performed by paired, one-tailed Student's t-test. (j) Neutrophil infiltration as measured by flow cytometry in the spleen (left panel) or SI-LP (right panel) of WT mice 18 h post CD3 Ab injection (20 μg, i.p.). Error bars indicate SEM from n=4 independent experiments. (k) qPCR of Il1b mRNA in lysates of splenocytes collected from WT or Rag1^{-/-} mice 4 h post CD3 Ab injection (50 μg, i.v.). Data are normalized to Hprt. Error bars indicate SEM from n=3 technical replicates. Data are representative of two independent experiments. (l) Neutrophil infiltration in the spleen of Rag1^{-/-} mice as measured by flow cytometry 3-4 h post CD3 Ab injection (50 μg, i.v.). Error bars indicate SEM from n=3 independent experiments. (m) Expression of Foxp3 and (n) ICOS measured by flow cytometry in splenic live, CD4⁺ T cells from WT mice, 18 h post CD3 Ab injection (20 μg, i.p.). Error bars indicate SEM from n=3 independent experiments. (o) Infiltration of CD11b⁺ cells as measured by flow cytometry in the SI-LP or (p) spleen of WT and Il1b^{-/-} mice, 18 h post CD3 Ab injection (20 μg, i.p.). Error bars indicate SEM from n=3 independent experiments. (q) Expression of cell surface Fas measured by flow cytometry in CD11b⁺ or CD11c⁺ BMDCs from given genotypes. Data are representative of two independent experiments. (r) Neutrophil infiltration (live, CD11b⁺F480⁻) as measured by flow cytometry in the SI-LP of Fast or Fas^{fl/fl}×CD11c-cre (Fas^{ΔDC}) mice, 18 h post CD3 Ab injection (20 μg, i.p.). Error bars indicate SEM from n=5 independent experiments. Splenocytes were taken from WT mice immunized with MOG35-55, and stimulated for 4 d in vitro with MOG in the presence of IL-1β, IL-23, and anti-IFNγ. (s) IL-1β was quantified by ELISA in the supernatants of WT BMDCs cultured with CD4⁺ T cells isolated from the stimulated splenocyte culture in the presence of MOG₃₅₋₅₅ and neutralizing antibodies TNF Ab (20 μg/mL) or FasL Ab (10 μg/mL) for 24 h. Error bars indicate SEM from n=2 independent experiments. (j-p, r) Statistical analysis was performed by unpaired, one-tailed Student's t-test. *p<0.05, **p<0.01, ***p<0.001, ****p<0.0001, n.s.=not significant.

FIG. 7: TNFR and Fas signaling pathways, not Caspase-1, is responsible for IL-1β mediated autoimmune inflammation. (a) IL-1β was quantified by ELISA in the supernatants of T_{H}17 polarized 2D2 TCR Tg cells cultured with WT BMDCs and MOG₃₅₋₅₅ (30 μM) in the presence of neutralizing TNF Ab (20 μg/mL) or FasL Ab (10 μg/mL) for 18 h. Error bars indicate SEM from n=5 independent experiments. Statistical analysis was performed by paired, one-tailed Student's t-test. *p<0.05. (b) Mean EAE clinical disease scores of WT, Il1b^{-/-}, and Casp1^{-/-} recipients after adoptive transfer of WT CD4⁺ T cells primed in vivo with MOG₃₅₋₅₅. Error bars indicate SEM from n=4 individual mice. Statistical analysis was performed by two-way repeated-measures ANOVA test, ****p<0.0001, n.s.=not significant. DF=16, F=0.0304 (WT vs Casp1^{-/-}), F=4.764 (WT vs Il1b^{-/-}) (c) Mean EAE clinical disease scores of WT, Tnfrsf1a^{-/-}Tnfrsf1b^{-/-}, or lpr recipients after adoptive transfer of WT CD4⁺ T cells primed in vivo with MOG₃₅₋₅₅. Error bars indicate SEM from n=5 individual mice. Statistical analysis was performed by two-way repeated-measures ANOVA test, ****p<0.0001, DF=14, F=7.136 (WT vs Tnfrsf1a^{-/-}Tnfrsf1b^{-/-}), F=5.417 (WT vs Lpr). (d) Percentage of mice with given disease scores in each recipient group from (c). (e) Luxol fast blue staining of spinal cords from given genotypes, 28 days after CD4⁺ T cell transfer, was performed to assess demyelination. Scale bar is 100 μm. Data are representative of two independent experiments.

(f) Illustration of “T cell-instructed” IL-1b production by MPs and its comparison to inflammasome induced IL-1b production by macrophages. (Left) During inflammasome activation in monocytes and macrophages, TLR and NLR-caspase-1 activation leads to synthesis and cleavage of pro-IL-1β, respectively. Robust production of IL-1b as a result of inflammasome activation can sometimes be critical for pathogen clearance. (Right) In contrast, “T cell-instructed” IL-1β production by antigen presenting MPs utilizes TNFR signaling for pro-IL1β synthesis while the cleavage signal is provided by Fas-caspase-8 axis. The IL-1β produced upon such T cell instruction drives cytokine production by effector CD4 T cells, systemic leukocyte recruitment, and autoimmunity. Figure was created using Biorender.

FIG. 8: (a) Enlargement of spleen and lymph nodes. (b) Spleen cell counts from multiple mice. (c) Serum cytokines from the mice on day 5 of DT injection. (d) and (e) Treg depletion leads to organ damage.

FIG. 9: The role of TNFR signaling and CD40 signaling to induce Inflammation and auto-immunity. (a) and (b) Interaction of effector CD4 T cells with Dendritic cells leads to production of inflammatory cytokines by Dendritic cells. (c) The inflammatory cytokine production induced by CD4 T cells in DCs is inhibited by blocking TNF and CD40 signaling. (d) In vivo injection of ant-CD3 (50 μg/mouse) leads to Cytokine storm/inflammatory cytokine production and (e) that is inhibited by blocking TNF and CD40. (f) Anti-CD3 injection into mice (200 μg/mouse) leads to cytokine storm induced mortality but mice are protected by blocking TNF and CD40 (blockade).

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include methods for treating an animal for a disease comprising one or more administrations of one or more compositions comprising (a) a TNF signaling inhibitor, (b) a CD40 inhibitor, a FAS signaling inhibitor, or both, and (c) optionally, a caspase 8 inhibitor. Other embodiments include methods for treating the disease comprising one or more administrations of one or more compositions comprising (a) the TNF signaling inhibitor and (b) the CD40 inhibitor. Certain embodiments include methods for treating the disease comprising one or more administrations of one or more compositions comprising (a) the TNF signaling inhibitor, (b) the FAS signaling inhibitor, and (c) optionally, the caspase 8 inhibitor. Still other embodiments include methods for treating a human for autoimmune disease, T cell mediated autoimmune disease, IL-1β mediated autoimmune disease, or cytokine release syndrome. Additional embodiments of the invention are also discussed herein.

Treatments of Disease

Some embodiments of the invention include treatment of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) by administering an inhibitor composition comprising (a) a TNF signaling inhibitor, (b) a CD40 inhibitor, a FAS signaling inhibitor, or both and (c) optionally a caspase 8 inhibitor. The inhibitor composition can be administered to animals by any number of suitable administration routes or formulations. The inhibitor composition can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

Some embodiments of the invention include treatment of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) by administering the inhibitor composition comprising (a) a TNF signaling inhibitor and (b) a CD40 inhibitor. The inhibitor composition can be administered to animals by any number of suitable administration routes or formulations. The inhibitor composition can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats.

Some embodiments of the invention include treatment of disease (e.g., autoimmune disease or transplant rejection) by administering the inhibitor composition comprising (a) a TNF signaling inhibitor, (b) a FAS signaling inhibitor and (c) optionally a caspase 8 inhibitor. The inhibitor composition can be administered to animals by any number of suitable administration routes or formulations. The inhibitor composition can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats.

The route of administration of the inhibitor composition can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intratracheal administration, intranasal administration, or intramuscular administration. In some embodiments, the administration can be an intratracheal administration, intranasal administration, an aerosol administration, a nebulizer administration, a pressurized metered-dose inhaler (pMDI) administration, an inhaler administration, or a dry powder inhaler (DPI) administration. In other embodiments, the administration can be an intravenous administration, a depot injection, a subcutaneous administration, a topical administration, an oral administration, a sublingual administration, or an intramuscular administration. The choice of administration route can depend on the compound identity (e.g., the physical and chemical properties of the one or more of the TNF signaling inhibitor, the CD40 inhibitor, the FAS signaling inhibitor, or the caspase 8 inhibitor) as well as the age and weight of the animal, the particular disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome), and the severity of the disease (e.g., stage or severity of autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). Of course, combinations of administration routes can be administered, as desired.

Some embodiments of the invention include a method for providing a subject with any inhibitor composition described herein (e.g., a pharmaceutical composition) which comprises one or more administrations of one or more such inhibitor compositions; the inhibitor compositions may be the same or different if there is more than one administration.

Diseases that can be treated in an animal (e.g., mammals, porcine, canine, avian (e.g., chicken), bovine, feline, primates, rodents, monkeys, rabbits, mice, rats, and humans) using any composition disclosed herein (e.g., an inhibitor composition) include, but are not limited to autoimmune diseases, T cell mediated autoimmune diseases, IL-1β mediated autoimmune diseases, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis or cytokine release syndrome (e.g., caused by viral infections or CAR T cell therapy). In certain embodiments, T cell mediated autoimmune diseases include but are not limited to type 1 diabetes, rheumatoid arthritis, multiple sclerosis, and Celiac disease. In some embodiments, IL-1β mediated autoimmune diseases include but are not limited to type 1 diabetes, pericarditis, rheumatoid arthritis, and psoriasis.

In some embodiments, diseases that can be treated in an animal (e.g., mammals, porcine, canine, avian (e.g., chicken), bovine, feline, primates, rodents, monkeys, rabbits, mice, rats, and humans) using the inhibitor composition include, but are not limited to autoimmune disease, T cell mediated autoimmune disease, IL-1β mediated autoimmune disease, type 1 diabetes, pericarditis, rheumatoid arthritis, psoriasis, psoriatic arthritis, ankylosing spondylitis, juvenile rheumatoid arthritis, multiple sclerosis, inflammatory bowel disease, Crohn's disease, ulcerative colitis, graft vs. host disease, transplant rejection, or cytokine release syndrome (e.g., caused by viral infections or CAR T cell therapy).

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease, autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, or cytokine release syndrome).

In some embodiments, diseases (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) that can be treated in an animal (e.g., mammals, porcine, canine, avian (e.g., chicken), bovine, feline, primates, rodents, monkeys, rabbits, mice, rats, and humans) using the inhibitor composition include, but are not limited to diseases (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) that can be treated by inhibiting (e.g., reducing the activity or expression of) one or more of TNF signaling inhibitor, TNF, FAS signaling, FAS, and caspase 8. In some embodiments, diseases (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) that can be treated in an animal (e.g., mammals, porcine, canine, avian (e.g., chicken), bovine, feline, primates, rodents, monkeys, rabbits, mice, rats, and humans) using the inhibitor composition include, but are not limited to diseases (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) that can be treated by reducing IL-1β production (e.g., via T cell instruction).

As used herein, the term “treating” (and its variations, such as “treatment”) is to be considered in its broadest context. In particular, the term “treating” does not necessarily imply that an animal is treated until total recovery. Accordingly, “treating” includes amelioration of a symptom, relief from a symptom or effect associated with a condition, decrease in severity of a condition, or preventing, preventively ameliorating a symptom, or otherwise reducing the risk of developing a particular condition. As used herein, reference to “treating” an animal includes but is not limited to prophylactic treatment (e.g., to prevent cytokine release syndrome) and therapeutic treatment. Any of the compositions (e.g., pharmaceutical compositions) described herein (e.g., an inhibitor composition) can be used to treat an animal.

As related to treating disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome), treating can include but is not limited to prophylactic treatment and therapeutic treatment. As such, treatment can include, but is not limited to: preventing disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); reducing the risk of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); ameliorating or relieving a symptom of a disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); eliciting a bodily response against disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome);

inhibiting the development or progression of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); inhibiting or preventing the onset of a symptom associated with disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); reducing the severity of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); causing a regression of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) or one or more symptoms associated with disease (e.g., inflammation); causing remission of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome); or preventing relapse of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In some embodiments, treating does not include prophylactic treatment of disease (e.g., preventing or ameliorating future disease).

Treatment of an animal (e.g., human) can occur using any suitable administration method (such as those disclosed herein) and using any suitable inhibitor composition (e.g., comprising a TNF signaling inhibitor and a CD40 inhibitor or comprising a TNF signaling inhibitor, a FAS signaling inhibitor and optionally a caspase 8 inhibitor). In some embodiments, methods of treatment comprise treating an animal for disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). Some embodiments of the invention include a method for treating a subject (e.g., an animal such as a human or primate) with an inhibitor composition (e.g., comprising a TNF signaling inhibitor and a CD40 inhibitor or comprising a TNF signaling inhibitor, a FAS signaling inhibitor and optionally a caspase 8 inhibitor) (e.g., a pharmaceutical composition) which comprises one or more administrations of one or more such inhibitor compositions; the inhibitor compositions may be the same or different if there is more than one administration.

In some embodiments, the method of treatment includes administering an effective amount of the components of an inhibitor composition (e.g., comprising a TNF signaling inhibitor and a CD40 inhibitor or comprising a TNF signaling inhibitor, a FAS signaling inhibitor and optionally a caspase 8 inhibitor). As used herein, the term “effective amount” refers to a dosage or a series of dosages sufficient to affect treatment (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of one or more components (e.g., the TNF signaling inhibitor, the CD40 inhibitor, the FAS signaling inhibitor, or the caspase 8 inhibitor) in the inhibitor composition (e.g., comprising a TNF signaling inhibitor and a CD40 inhibitor or comprising a TNF signaling inhibitor, a FAS signaling inhibitor and optionally a caspase 8 inhibitor) (which can be administered to an animal such as mammals, primates, monkeys or humans) can be an amount of about 0.005 to about 50 mg/kg body weight, about 0.005 to about 80 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, an effective amount of one or more components (e.g., the TNF signaling inhibitor, the CD40 inhibitor, the FAS signaling inhibitor, or the caspase 8 inhibitor) in the inhibitor composition (e.g., comprising a TNF signaling inhibitor and a CD40 inhibitor or comprising a TNF signaling inhibitor, a FAS signaling inhibitor and optionally a caspase 8 inhibitor) (which can be administered to an animal such as mammals, rodents, mice, rabbits, feline, porcine, or canine) can be an amount of about 0.005 to about 50 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of one or more components (e.g., the TNF signaling inhibitor, the CD40 inhibitor, the FAS signaling inhibitor, or the caspase 8 inhibitor) in the inhibitor composition (e.g., comprising a TNF signaling inhibitor and a CD40 inhibitor or comprising a TNF signaling inhibitor, a FAS signaling inhibitor and optionally a caspase 8 inhibitor) (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some embodiments, the dosage can be about 5 mg/kg human body weight, about 10 mg/kg human body weight, about 20 mg/kg human body weight, about 80 mg/kg human body weight, or about 100 mg/kg human body weight. In some instances, an effective amount of one or more components (e.g., the TNF signaling inhibitor, the CD40 inhibitor, the FAS signaling inhibitor, or the caspase 8 inhibitor) in the inhibitor composition (e.g., comprising a TNF signaling inhibitor and a CD40 inhibitor or comprising a TNF signaling inhibitor, a FAS signaling inhibitor and optionally a caspase 8 inhibitor) (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 80 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

“Therapeutically effective amount” means an amount effective to achieve a desired and/or beneficial effect (e.g., decreasing a symptom of a disease). A therapeutically effective amount can be administered in one or more administrations. For some purposes of this invention, a therapeutically effective amount is an amount appropriate to treat an indication (e.g., to treat autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, or cytokine release syndrome). By treating an indication is meant achieving any desirable effect, such as one or more of palliate, ameliorate, stabilize, reverse, slow, or delay disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) progression, increase the quality of life, or to prolong life. Such achievement can be measured by any suitable method, such as but not limited to (a) measurement of the amount of one or more of antibodies (e.g., related to a particular disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome)), anti-dsDNA, anti-RNP, anti-Smith (or anti-Sm), anti-Sjogren's SSA and SSB, anti-scleroderma, anti-Scl-70, anti-Jo-1, anti-CCP Antibody against cardiolipin, rheumatoid factor antibodies, IL-12, IL-6, or IL-1β; (b) measurement of the amount of one or more cytokines; (c) using an antinuclear antibody test; (d) assessing the state of a transplanted organ, (e) using an EAE clinical disease score, (f) using an MS clinical disease score, (g) assessing liver injury or size, (h) assessing lung injury or size, (i) assessing spleen injury or size, (j) assessing lymph node injury or size, (k) assessing inflammation, or (l) any suitable method to assess the progression of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, other disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) treatments are optionally included, and can be used with the inventive treatments described herein (e.g., administering inhibitor composition). Other disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) treatments can include any known treatment that is suitable to treat the disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, additional optional treatments (e.g., as an other treatment of autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome) can also include one or more of surgical intervention, hormone therapies, immunotherapy, and adjuvant systematic therapies.

TNF (Tumor Necrosis Factor) Signaling Inhibitors

In some embodiments of the invention, any suitable inhibitor composition comprising a TNF signaling inhibitor (e.g., TNFR inhibitor (e.g., TNFR1 inhibitor or TNFR2 inhibitor) or TNF inhibitor (e.g., TNFα inhibitor)) can be used in the methods described herein, including but not limited to methods for treating disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the TNF signaling inhibitor (e.g., TNFR inhibitor (e.g., TNFR1 inhibitor or TNFR2 inhibitor) or TNF inhibitor (e.g., TNFα inhibitor)) can inhibit (e.g., fully inhibit or partially inhibit) one or more TNF signals (e.g., via a TNFR (e.g., TNFR1 or TNFR2) or a TNF (e.g., TNFα)) by, for example, reducing the activity or expression of TNFR (e.g., TNFR1 or TNFR2) or TNF (e.g., TNFα)). In other embodiments, the TNF signaling inhibitor (e.g., TNFR inhibitor (e.g., TNFR1 inhibitor or TNFR2 inhibitor) or TNF inhibitor (e.g., TNFα inhibitor)) can be TNFR antagonists, TNFR1 antagonists, TNFR2 antagonists, TNF antagonists, TNFα antagonists, TNFR partial antagonists, TNFR1 partial antagonists, TNFR2 partial antagonists, TNF partial antagonists, TNFα partial antagonists, TNFR inverse agonists, TNFR1 inverse agonists, TNFR2 inverse agonists, TNF inverse agonists, TNFα inverse agonists, TNFR partial inverse agonists, TNFR1 partial inverse agonists, TNFR2 partial inverse agonists, TNF partial inverse agonists, or TNFα partial inverse agonists, or combinations thereof. In certain embodiments, inhibition can occur using any suitable mechanism, such as but not limited to blockading a receptor (e.g., partially or fully blocking other molecules from accessing one or more receptor sites), an antagonist mechanism, a partial antagonist mechanism, an inverse agonist mechanism, a partial inverse agonist mechanism, or a combination thereof.

In some embodiments, TNF signaling that can be inhibited can include any suitable aspect of TNF signaling (e.g., by inhibiting TNFR, TNFR1, TNFR2, TNF, or TNFα) that can be inhibited to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In other embodiments, the TNF signaling inhibitor can, in some embodiments, inhibit one or more of the following: TNFR, TNFR1, TNFR2, TNF, or TNFα.

In some embodiments, the TNF signaling inhibitor (e.g., TNFR inhibitor (e.g., TNFR1 inhibitor or TNFR2 inhibitor) or TNF inhibitor (e.g., TNFα inhibitor)) can include any suitable TNF signaling inhibitor to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In other embodiments, the TNF signaling inhibitor can be, but is not limited to, a TNF signaling antagonist, a TNF signaling partial antagonist, a TNF signaling inverse agonist, or a TNF signaling partial inverse agonist, or a combination thereof.

In some embodiments, the TNF signaling inhibitor (e.g., TNFR inhibitor (e.g., TNFR1 inhibitor or TNFR2 inhibitor) or TNF inhibitor (e.g., TNFα inhibitor)) can be protein, an oligopeptide, an siRNA, an antibody, a bispecific antibody, a monoclonal antibody, a small molecule (e.g., small organic molecule), infliximab (Remicade) and biosimilars thereof, adalimumab (Humira) and biosimilars thereof, certolizumab and biosimilars thereof, certolizumab pegol (Cimzia) and biosimilars thereof, golimumab (Simponi) and biosimilars thereof, etanercept (Enbrel) and biosimilars thereof, Remsima, Thalidomide (2-(2,6-dioxopiperidin-3-yl)-2,3-dihydro-1H-isoindole-1,3-dione; CAS #50-35-1) and its derivatives (e.g. esters, ethers, or amides), lenalidomide ((3RS)-3-(4-Amino-1-oxo-1,3-dihydro-2H-isoindo1-2-yl)piperidine-2,6-dione; CAS #191732-72-6), pomalidomide (4-amino-2-(2,6-dioxopiperidin-3-yl)isoindole-1,3-dione; CAS #19171-19-8), xanthine (3,7-dihydropurine-2,6-dione) derivatives (e.g. pentoxifylline which is 3,7-Dimethyl-1-(5-oxohexyl)-3,7-dihydro-1H-purine-2,6-dione and CAS #6493-05-6), bupropion ((RS)-2-(tert-Butylamino)-1-(3-chlorophenyl)propan-1-one; CAS #34911-55-2), 5-HT2A agonist (e.g., uniprot P28223 (human) and P35363 (mouse)), (R)-DOI (1-(4-Iodo-2,5-dimethoxyphenyl)propan-2-amine), TCB-2 ([(7R)-3-Bromo-2,5-dimethoxy-bicyclo[4.2.0]octa-1,3,5-trien-7-yl]methanamine; CAS #912440-88-1), LSD ((6aR,9R)-N,N-diethyl-7-methyl-4,6,6a,7,8,9-hexahydroindolo[4,3-fg]quinoline-9-carboxamide; CAS #50-37-3), LA-SS-Az ([(6aR,9R)-7-methyl-6,6a,8,9-tetrahydro-4H-indolo[4,3-fg]quinoline-9-yl]-[(2S,4S)-2,4-dimethylazetidin-1-yl]methanone; CAS #470666-31-0), curcumins (e.g., (1E,6E)-1,7-Bis(4-hydroxy-3-methoxyphenyl)hepta-1,6-diene-3,5-dione; CAS #458-37-7), catechins ((2R,3S)-2-(3,4-Dihydroxyphenyl)-3,4-dihydro-2H-chromene-3,5,7-triol; CAS #154-23-4), Cannabidiol (2-[(1R,6R)-6-Isopropenyl-3-methylcyclohex-2-en-1-yl]-5-pentylbenzene-1,3-diol; CAS #13956-29-1) or the chemical constituents or extracts of Echinacea purpurea. In other embodiments, the TNF signaling inhibitor is not a CD40 inhibitor.

CD40 (Cluster of Differentiation 40) Inhibitors

In some embodiments of the invention, any suitable inhibitor composition comprising a CD40 inhibitor (e.g., a CD40L inhibitor, a CD40 antibody or an anti-CD40L antibody) can be used in the methods described herein, including but not limited methods for treating disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the CD40 inhibitor (e.g., a CD40L inhibitor, a CD40 antibody or an anti-CD40L antibody) can inhibit (e.g., fully inhibit or partially inhibit) one or more CD40s by, for example, reducing the activity or expression of CD40 or CD40L. In other embodiments, the CD40 inhibitor (e.g., a CD40L inhibitor, a CD40 antibody or an anti-CD40L antibody) can be CD40 antagonists, CD40 partial antagonists, CD40 inverse agonists, CD40 partial inverse agonists, or combinations thereof. In other embodiments, the CD40L inhibitor (e.g., an anti-CD40L antibody) can be CD40L antagonists, CD40L partial antagonists, CD40L inverse agonists, CD40L partial inverse agonists, or combinations thereof. In certain embodiments, inhibition can occur using any suitable mechanism, such as but not limited to blockading a receptor (e.g., partially or fully blocking other molecules from accessing one or more receptor sites), an antagonist mechanism, a partial antagonist mechanism, an inverse agonist mechanism, a partial inverse agonist mechanism, or a combination thereof.

In some embodiments, CD40 that can be inhibited can include any suitable aspect of CD40 (e.g., CD40L) that can be inhibited to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the CD40 inhibitor (e.g., a CD40L inhibitor, a CD40 antibody or an anti-CD40L antibody) can include any suitable CD40 inhibitor to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In other embodiments, the CD40 inhibitor can be, but is not limited to, a CD40 antagonist, a CD40 partial antagonist, a CD40 inverse agonist, or a

CD40 partial inverse agonist, or a combination thereof. In other embodiments, the CD40 inhibitor can be, but is not limited to, a CD40L antagonist, a CD40L partial antagonist, a CD40L inverse agonist, or a CD40L partial inverse agonist, or a combination thereof.

In some embodiments, the CD 40 inhibitor (e.g., a CD40 antibody or an anti-CD40L antibody) can be protein, an oligopeptide, an siRNA, an antibody, a bispecific antibody, a monoclonal antibody, a small molecule (e.g., small organic molecule), a CD40 antibody, an anti-CD40L antibody (e.g., anti-CD40L Ab (BioLegend 310812)), BI 655064 (AbbVie), CFZ533 (iscalimab)(Novartis), BG9588 (Biogen, Inc.), or KPL-404 (anti-CD40 blocking antibody, Kiniska Pharmaceuticals, <<www.kiniska.com>>).

FAS (Fas Receptor) Signaling Inhibitors

In some embodiments of the invention, any suitable inhibitor composition comprising a FAS signaling inhibitor (e.g., a FAS inhibitor or an anti-FAS antibody) can be used in the methods described herein, including but not limited methods for treating disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the FAS signaling inhibitor (e.g., a FAS inhibitor or an anti-FAS antibody) can inhibit (e.g., fully inhibit or partially inhibit) one or more FAS signals (e.g., via FAS) by, for example, reducing the activity or expression of FAS. In other embodiments, FAS signaling inhibitors (e.g., a FAS inhibitor or an anti-FAS antibody) can be FAS antagonists, FAS partial antagonists, FAS inverse agonists, FAS partial inverse agonists, or combinations thereof. In certain embodiments, inhibition can occur using any suitable mechanism, such as but not limited to blockading a receptor (e.g., partially or fully blocking other molecules from accessing one or more receptor sites), an antagonist mechanism, a partial antagonist mechanism, an inverse agonist mechanism, a partial inverse agonist mechanism, or a combination thereof.

In some embodiments, FAS signaling that can be inhibited can include any suitable aspect of FAS signaling (e.g., by inhibiting FAS) that can be inhibited to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In other embodiments, the FAS signaling inhibitor can, in some embodiments, inhibit one or more of FAS.

In some embodiments, the FAS signaling inhibitor (e.g., a FAS inhibitor or an anti-FAS antibody) can include any suitable FAS signaling inhibitor to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In other embodiments, the FAS signaling inhibitor can be, but is not limited to, a FAS signaling antagonist, a FAS signaling partial antagonist, a FAS signaling inverse agonist, or a FAS signaling partial inverse agonist, or a combination thereof.

In some embodiments, the FAS signaling inhibitor (e.g., a FAS inhibitor or an anti-FAS antibody) can be protein, an oligopeptide, an siRNA, an antibody, a bispecific antibody, a monoclonal antibody, a small molecule (e.g., small organic molecule), or anti-FasL Ab (BioLegend 106608).

Caspase 8 Inhibitors

In some embodiments of the invention, any suitable inhibitor composition comprising a caspase 8 inhibitor (e.g., Emricasan) can be used in the methods described herein, including but not limited methods for treating disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the caspase 8 inhibitor (e.g., Emricasan) can inhibit (e.g., fully inhibit or partially inhibit) caspase 8 by, for example, reducing the activity or expression of caspase 8. In other embodiments, caspase 8 inhibitor (e.g., Emricasan) can be caspase 8 antagonists, caspase 8 partial antagonists, caspase 8 inverse agonists, caspase 8 partial inverse agonists, or combinations thereof. In certain embodiments, inhibition can occur using any suitable mechanism, such as but not limited to blockading a receptor (e.g., partially or fully blocking other molecules from accessing one or more receptor sites), an antagonist mechanism, a partial antagonist mechanism, an inverse agonist mechanism, a partial inverse agonist mechanism, or a combination thereof.

In some embodiments, the caspase 8 activity that can be inhibited can include any suitable aspect of caspase 8 activity that can be inhibited to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the caspase 8 inhibitor (e.g., Emricasan) can include any suitable caspase 8 inhibitor to treat disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In other embodiments, the caspase 8 inhibitor can be, but is not limited to, a caspase 8 antagonist, a caspase 8 partial antagonist, a caspase 8 inverse agonist, or a caspase 8 partial inverse agonist, or a combination thereof.

In some embodiments, the caspase 8 inhibitor can be a protein, an oligopeptide, an siRNA, an antibody, a bispecific antibody, a monoclonal antibody, a small molecule (e.g., small organic molecule), or Emricasan ((3S)-3-{[(2S)-2-{[2-(2-tert-butylanilino)-2-oxoacetyl]amino}propanoyl]amino }-4-oxo-5-(2,3,5,6-tetrafluorophenoxy)pentanoic acid; CAS #254750-02-2).

Compositions Used for Treating

In certain embodiments, one or more components (e.g., the TNF signaling inhibitor, the CD40 inhibitor, the FAS signaling inhibitor, or the caspase 8 inhibitor) of the inhibitor composition (e.g., comprising a TNF signaling inhibitor and a CD40 inhibitor or comprising a TNF signaling inhibitor, a FAS signaling inhibitor and optionally a caspase 8 inhibitor) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

In some embodiments, one or more components (e.g., the TNF signaling inhibitor, the CD40 inhibitor, the FAS signaling inhibitor, or the caspase 8 inhibitor) of the inhibitor composition (e.g., comprising a TNF signaling inhibitor and a CD40 inhibitor or comprising a TNF signaling inhibitor, a FAS signaling inhibitor and optionally a caspase 8 inhibitor) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising one or more components (e.g., the TNF signaling inhibitor, the CD40 inhibitor, the FAS signaling inhibitor, or the caspase 8 inhibitor) of the inhibitor composition (e.g., comprising a TNF signaling inhibitor and a CD40 inhibitor or comprising a TNF signaling inhibitor, a FAS signaling inhibitor and optionally a caspase 8 inhibitor). In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

An effective amount (e.g., a therapeutically effective amount) can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as but not limited to (a) measurement of the amount of one or more of antibodies (e.g., related to a particular disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome)), anti-dsDNA, anti-RNP, anti-Smith (or anti-Sm), anti-Sjogren's SSA and SSB, anti-scleroderma, anti-Scl-70, anti-Jo-1, anti-CCP Antibody against cardiolipin, rheumatoid factor antibodies, IL-12, IL-6, or IL-1β; (b) measurement of the amount of one or more cytokines; (c) using an antinuclear antibody test; (d) assessing the state of a transplanted organ, (e) using an EAE clinical disease score, (f) using an MS clinical disease score, (g) assessing liver injury or size, (h) assessing lung injury or size, (i) assessing spleen injury or size, (j) assessing lymph node injury or size, (k) assessing inflammation, or (l) any suitable method to assess the progression of disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, one or more components (e.g., the TNF signaling inhibitor, the CD40 inhibitor, the FAS signaling inhibitor, or the caspase 8 inhibitor) of the inhibitor composition (e.g., comprising a TNF signaling inhibitor and a CD40 inhibitor or comprising a TNF signaling inhibitor, a FAS signaling inhibitor and optionally a caspase 8 inhibitor) can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, depot injection (e.g., solid or oil based), subcutaneous administration, topical administration, intradermal administration, oral administration, sublingual administration, intratracheal administration, intranasal administration, or intramuscular administration. In some embodiments, the pharmaceutical composition can be presented in a dosage form which is suitable for an intratracheal administration, an intranasal administration, an aerosol administration, a nebulizer administration, a pressurized metered-dose inhaler (pMDI) administration, an inhaler administration, or a dry powder inhaler (DPI) administration. The pharmaceutical composition can be in any suitable form, for example but not limited to, tablets, capsules, pills, powders, granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. For example, a depot injection (e.g., solid or oil based) could include one or more formulary ingredients.

In certain embodiments, pharmaceutical compositions can be formulated to release one or more components (e.g., the TNF signaling inhibitor, the CD40 inhibitor, the FAS signaling inhibitor, or the caspase 8 inhibitor) of the inhibitor composition (e.g., comprising a TNF signaling inhibitor and a CD40 inhibitor or comprising a TNF signaling inhibitor, a FAS signaling inhibitor and optionally a caspase 8 inhibitor) substantially immediately upon the administration or any substantially predetermined time or time after administration. Such formulations can include, for example, controlled release formulations such as various controlled release compositions and coatings. For example, a depot injection (e.g., solid or oil based) could be used for a controlled release, and in some instances, could be injected once per month (or once per day, once per week, once per three months, once per six months, or once per year).

Other formulations (e.g., formulations of a pharmaceutical composition) can, in certain embodiments, include those incorporating the drug (or control release formulation) into food, food stuffs, feed, or drink. For example, the inhibitor composition could be administered orally once per day, twice per day, three times per day, once per two days, or once per week.

Some embodiments of the invention can include methods of treating an organism for disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome). In certain embodiments, treating comprises administering at least one inhibitor composition. In other embodiments, treating comprises administering at least one inhibitor composition to an animal that is effective to treat autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, or cytokine release syndrome. In some embodiments, a composition or pharmaceutical composition comprises one or more components (e.g., the TNF signaling inhibitor, the CD40 inhibitor, the FAS signaling inhibitor, or the caspase 8 inhibitor) of the inhibitor composition (e.g., comprising a TNF signaling inhibitor and a CD40 inhibitor or comprising a TNF signaling inhibitor, a FAS signaling inhibitor and optionally a caspase 8 inhibitor) which can be administered to an animal (e.g., mammals, primates, monkeys, or humans) in an amount of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, some animals (e.g., mammals, mice, rabbits, feline, porcine, or canine) can be administered a dosage of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. It is possible to employ many concentrations in the methods of the present invention, and using, in part, the guidance provided herein, one would be able to adjust and test any number of concentrations in order to find one that achieves the desired result in a given circumstance. In other embodiments, the inhibitor composition can be administered in combination with one or more other therapeutic agents to treat a given disease (e.g., autoimmune diseases, T cell mediated autoimmune diseases, IL-1β mediated autoimmune diseases, transplant rejection, type 1 diabetes, rheumatoid arthritis, multiple sclerosis, graft vs. host disease, Celiac disease, pericarditis, psoriasis, uveitis, and cytokine release syndrome).

In some embodiments, the compositions can include a unit dose of one or more inhibitor compositions in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, lactose powder or saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, lactose powder or the like, and combinations thereof. Nontoxic auxiliary substances, such as wetting agents, buffers, or emulsifiers may also be added to the composition. Formulations (e.g., oral formulations) can include such normally employed excipients as, for example, pharmaceutical grades of mannitol, lactose, starch, magnesium stearate, sodium saccharine, cellulose, and magnesium carbonate.

The presently-disclosed subject matter is further illustrated by the following specific but non-limiting examples. The following examples may include compilations of data that are representative of data gathered at various times during the course of development and experimentation related to the present invention.

### EXAMPLES

### Example Set 1

Some aspects of this Example Set are related to Jain et al. (2020) “T cells instruct myeloid cells to produce inflammasome-independent IL-1β and cause autoimmunity” Nature Immunology, Vol. 21, pp. 65-74. Related material such as abbreviations, can be found in that document.

Due to its highly inflammatory nature, IL-1β is produced under regulation in a two-step mechanism. The transcription and translation of pro-IL-1β, which is dependent on the activation of the transcription factor NF-κB is induced by the activation of pattern recognition receptors (PRRs) such as the Toll-like receptors (TLRs). Because pro-IL-1β is not biologically active, it requires the proteolytic cleavage of pro-IL-1β into its bioactive form. Activation of the inflammasomes by damage-associated molecules or microbial virulence factors induces the casp-1-dependent processing of pro-IL-1β.

Here, we investigated how bioactive IL-1β was produced during T cell-driven autoimmune diseases in the absence of overt infection or injury. We describe a mechanism of IL-1β production that is independent of signaling through PRRs and inflammasome activation. We found that during cognate interaction, effector-memory CD4⁺ T cells instructed antigen-presenting myeloid cells to produce mature IL-1β. This T cell-induced IL-1β was dependent on the expression of the cytokine TNF and the membrane-bound protein FasL by the activated T cells during their interaction with the macrophages or DCs (hereafter, mononuclear phagocytes, MPs). Signaling through the TNF receptor (TNFR) appeared to be required for the synthesis of pro-IL-1β in MPs. The interaction with activated T cells also triggered signaling through the surface receptor for FasL, Fas, in MPs, which resulted in casp-8-dependent maturation of pro-IL-1β. This TNFR-Fas pathway of IL-1β production was responsible for the induction of inflammation and pathology during experimental autoimmune encephalomyelitis (EAE), a T cell-mediated autoimmune disease, suggesting this pathway was likely responsible for the production of IL-1β during T cell-driven autoimmune pathology.

Methods

Mice

C57BL/6 wild-type control mice were obtained from the UT Southwestern Mouse Breeding Core Facility. IL-1β^{-/-} mice were generated by David Chaplin, UA at Birmingham and provided to us by Fayyaz S. Sutterwala at Cedars Sinai. Rip3^{-/-} and Rip3^{-/-}Casp8^{-/-} mice were provided by Andrew Oberst at the University of Washington. ASC KO mice were a gift from Genentech. Gsdmd^{-/-} mice were provided by Jonathan Kagan at Boston Children's Hospital. Caspase-1 KO were provided by Russell Vance at University of California, Berkeley. B6.MRL-Fas^{lpr}/J (lpr), B6.129S-Tnf^{tm1Gkl}/J (Tnfa^{-/-}), B6.129S-Tnfrsf1a^{tm1Imx} Tnfrsf1b^{tm1Imx}/J (Tnfrsf1a/b^{-/-}), Rag1^{tm1Mom}, Casp1/11^{null}, 2D2 TCR transgenic mice and Zbtb46^{gfp} were obtained from Jackson Laboratories. All mice were bred and housed in a specific pathogen-free facility at UT Southwestern Medical Center and Cincinnati Children's Hospital Medical Center. For isolation of steady state CD4 memory T cells, mice were housed in a conventional facility for 2-4 weeks before tissue isolation. All mouse experiments were done as per protocols approved by Institutional Animal Care and Use Committee (IACUC) at UT Southwestern Medical Center and Cincinnati Children's Hospital Medical Center.

Reagents

IL-1Ra (R&D Systems; 480-RM, 50 ng/mL), rTNFα (Peprotech; 315-01Am, 20 ng/mL), αCD3e (Biolegend; 100331 for in vitro assays and 100340 for in vivo assays), αTNF (Biolegend; 506332, 20 μg/mL), αFasL (Biolegend; 106608, 10 μg/mL), αCD40L (Biolegend; 310812, 10 μg/mL), IL-1β ELISA (R&D Systems; DY-401), TNFα ELISA(Biolegend; 510802 for capture and 506312 for detection), Naïve T cell Mojosort (Biolegend; 480040), total CD4⁺ T cell Mojosort (Biolegend; 480006), Zombie yellow (Biolegend; 423103), DAPI (Biolegend; 422801), IETD (R&D Systems; FMK007, 10 μM), rGMCSF (Biolegend; 576306), Anti-biotin beads (Miltenyi Biotec; 130-090-485), OVA₃₂₃₋₃₃₉ (Invivogen; vac-isq), LPS (Sigma, 100 ng/mL), ATP (Invivogen; tlrl-atpl, 5 nM), IL17a ELISA (Biolegend; 506902 for capture and 507002 for detection), EAE immunization kit (Hooke Laboratories, EK-2110) mouse MOG₃₅₋₅₅ (CSbiologicals, CS0681)

Antibodies for Flow Cytometry

Anti-mouse Pro-IL-1β APC (eBioscience, 17-7114-80; 1:500), Anti-mouse CD11b BV785 (Biolegend, 101243; 1:400), Anti-mouse CD11c FITC (Biolegend; 117306, 1:400), Anti-mouse CD115 PE (Biolegend; 135505, 1:400), Anti-mouse Ly6G FITC (Biolegend, 127605; 1:400), Ly6C BV711 (Biolegend, 12803; 1:400), Anti-mouse TNFα FITC (Biolegend, 506304, 1:1000), Anti-mouse F4/80 APC-eflour 780 (Invitrogen, 47-4801-80; 1:400), Anti-mouse CD90 Pacific blue (Biolegend, 105324; 1:400), Anti-mouse FasL PE (Biolegend, 106605; 1:100), Anti-mouse CD62L biotin (Biolegend, 104404; 1:200), Anti-mouse CD11c biotin (Biolegend,117304; 1:500), Rorgt PE (ebioscience, 12-6988-82, 1:100), Tbet Pacific Blue (Biolegend, 644807, 1:100), GATA3 APC (Biolegend, 653806, 1:20), ICOS APC (Biolegend, 313509, 1:400), Foxp3 AF488 (Biolegend, 126405, 1:100).

Cell Culture

Complete RPMI media (RPMI1640, Hyclone) supplemented with L-glutamine, penicillin-streptomycin, sodium pyruvate, β-mercaptoethanol (Sigma) was used throughout the experiments.

In Vitro Differentiation of Naïve CD4⁺ T Cells:

Cell culture treated plates were coated with αCD3 (5 μg mL⁻¹) and αCD28 (5 μg mL⁻¹) for 3-4 h at 37° C. CD4⁺CD62L^{hi} naïve CD4⁺ T cells were isolated from splenocytes using a Mojosort naïve CD4 T cell isolation kit according to manufacturer's protocol. Purified naïve T cells were plated in antibody-coated plates with appropriate polarizing conditions for 5 days. T cell were cultured in complete RPMI supplemented with 10% FCS. Cytokine cocktails for in vitro polarization: Thl — IL-12 (10 ng mL⁻¹, peprotech), IL-2 (50 U mL⁻¹, peprotech), αIL-4 (10 μg mL⁻¹, Biolegend); Th17-IL-6 (20 ng mL⁻¹, peprotech), hTGFβ (5 ng mL⁻¹, peprotech), αIL-4 (10 μg mL⁻¹, Biolegend), αIFNγ (10 μg mL⁻¹, Biolegend), IL-23 (20 ng mL⁻¹, Biolegend) and IL-1β (20 ng mL⁻¹, peprotech), Th2− IL-4 (4 ng mL⁻¹, peprotech), IL-2 (50 U mL⁻¹, Biolegend), and αIFNγ (10 μg mL⁻¹, Biolegend), Th0− IL-2 (50 U mL⁻¹). For co-culture experiments T cells were rested in 10% RPMI supplemented with IL-2 (10 U mL⁻¹) for 36 hrs before co-culture.

In Vitro Differentiation of Bone Marrow Derived Dendritic Cells and Retroviral Transduction

Mouse progenitors were isolated from bone marrow (femurs and tibias). Following RBC lysis, cells were plated at 0.75×10⁶ mL⁻¹ in BMDC media (5% FCS containing complete RPMI+1% rGMCSF (Biolegend, 100 ng mL⁻¹). Media was replaced on day 2 and day 4 and cells were harvested for experiments on day 5 by gently flushing each well. For sorting of BMDC subpopulations, total BMDC cells were stained with fluorescent antibodies and FACS sorted as shown (FIG. 5g) For retroviral transduction, following RBC lysis cells were plated at 10×10⁶ mL⁻¹ in 2 mL of retroviral supernatant containing 8 μL mL⁻¹ polybrene. Cells were spinfected at 2500 rpm, 32° C., for 90 minutes, then 3 mL of BMDC media was added to each well and cells were incubated overnight. The next morning, ˜70% of the media was removed from the wells and spinfection was repeated with fresh retroviral supernatant. On day 5 cells were harvested for experiments.

In Vitro Differentiation of Bone Marrow Derived Macrophages

Mouse progenitors were isolated from bone marrow (femurs and tibias). Following RBC lysis, cells were plated in BMDM media in TC treated culture dish (5% FCS containing complete RPMI+ 30% L929 supernatant). On day one, non-adherent cells were transferred to a non-treated petri dish. After 5-7 days of culture in BMDM media, macrophages were obtained from the dish by EDTA treatment.

Isolation of CD44^{hi}CD62L^{lo} Cells from Spleen and Lymph Nodes

Single cell suspension was obtained from spleen and peripheral lymph nodes. 4-8 mice were pooled for each experiment. CD4⁺ T cells were isolated using mouse CD4 T cells isolation kit (Biolegend) following manufacturer's instructions. Cells were then labeled with anti-CD62L biotin antibody followed by anti-biotin microbeads (Miltenyi). CD62L^{1o} cells were isolated using negative selection on AutoMacs. Approximately 95% cells were CD4⁺CD44^{hi}CD62L^{lo}.

Purification of Various Myeloid Cells Populations

Splenocytes or total BMDCs were labelled with anti-CD11c biotin antibody followed by anti-biotin microbeads (Miltenyi). CD11c⁺ cells were magnetically sorted using positive selection on AutoMacs. CD11c⁺ enriched cells were then sorted based on various markers as described in the FIG. 5g-j using Moflo cell sorter. Sorting strategy and post-sort purities are shown in FIG. 5g-j.

Co-Culture of DC and T Cells for Induction of IL-1β

1 million DCs were cultured with 4 million T cells in 12 well plate. DC-T cell interaction was triggered by adding either αCD3ε (αCD3, 200 ng mL⁻¹) or OVA₃₂₃₋₃₃₉ as described in the legends. In experiments measuring secreted IL-1β, IL-1R antagonist (50 ng mL⁻¹) was added 1 hr prior to DC stimulation to block IL-1β consumption. Co-cultures were also pretreated with neutralizing antibodies and inhibitor wherever described. Co-culture experiments were performed in complete RPMI supplemented with 10% FCS. For Western blot analysis of the culture supernatant cells were cultured in 1% FCS containing complete RPMI.

T Cell Re-Stimulation (In Vivo)

6-8 weeks old mice were treated with 20 μg αCD3 or PBS by i.p. injection. Lamina propria cells and splenocytes were isolated at given time points after stimulation followed by surface and intracellular staining.

Quantitative Real-Time PCR

RNA was isolated using Qiagen RNA extraction kit using manufacturer's protocol. cDNA was synthesized using Random primers and MMLV reverse transcriptase (Invitrogen; 2805-013). The QuantStudio 7 Flex Real-Time PCR System (ThermoFisher Scientific) was used to measure SyBR green (ThermoFisher Scientific) incorporation. All data is normalized to 18s. qPCR primers sequences are as follows:

Il1b:
Fwd-
(SEQ ID NO: 1)
5’TGTGCTCTGCTTGTGAGGTGCTG 3’,
Rev
(SEQ ID NO: 2)
5’CCCTGCAGCTGGAGAGTGTGGA3’
Hprt1:
Fwd-
(SEQ ID NO: 3)
5’ CAGTCCCAGCGTCGTGATTA-3’,
Rev-
(SEQ ID NO: 4)
5’ TGGCCTCCCATCTCCTTCAT-3’
18S:
Fwd-
(SEQ ID NO: 5)
5’ GTAACCCGTTGAACCCCATT,
Rev-
(SEQ ID NO: 6)
5’ CCATCCAATCGGTAGTAGCG

Immunoblot Analysis

Cells were lysed in 1× RIPA Buffer and protein was quantified using Pierce™ BCA Protein Assay Kit. Cell lysates where boiled in 1× Laemmli buffer at 95° C. for 10 mins. Cell lysates were separated by SDS-PAGE and transferred onto PVDF membranes. Blots were incubated with anti-IL1β [1:1000] (R&D AF-401-SP), anti-caspase8 [1:1000] (Enzo ALX-804-447-C100), anti-caspase1 [1:1000] (Genentech), anti-βtubulin [1:5000] (CST 2146S). As secondary antibodies, anti-rabbit-IgG-HRP (Biorad) [1:5000], anti-mouse-IgG-HRP and anti-goat-IgG-HRP [1:10000] (Jackson ImmunoResearch Laboratory) were used. Anti-β-actin (C4, Santa Cruz, 1:5000) was used as control. Western blot was develop using SuperSignal™ West Pico PLUS Chemiluminescent Substrate (Thermo Fisher) and ECL signal was recorded on X-Ray Films using a developer (Kodak).

Surface and Intracellular Staining and Flow Cytometry

Cells were stained with relevant antibodies for 30 min on ice and washed. For intracellular staining, Foxp3 staining buffer set (eBioscience; 00-5523-00) was used according to manufacturer's protocol. The stained cells were analyzed with BDLSRII or Novocyte (ACEA biosciences). For cytokine receptor staining, control refers to fluorescence minus one control. Data were analyzed with FlowJo 10 Software.

Isolation of Lamina Propria Lymphocytes

Small intestines were flushed with cold PBS and carefully cut longitudinally. 1-2 cm pieces of the tissue were digested twice with 2 mM EDTA buffer followed by 3 rounds of enzymatic digestion with Collagenase IV (10 μg/mL, Sigma) and DNase I (500 μg/mL, Sigma). Cell suspensions obtained after digestions were loaded on a 40-70% percoll gradient as described before.

Passive EAE Induction

9-10 weeks old WT female mice were immunized with MOG₃₅₋₅₅ emulsion obtained from the Hooke lab EAE immunization kit as per manufacturer's protocol. Mice were also injected with 80 ng Pertussis toxin intraperitoneally on day 0 and day 1. Total splenocytes were harvested 11-14 days after immunization and cultured in vitro with MOG (20 μg/mL), anti-IFNγ (10 μg/mL) and IL-23 (10 ng/mL). After 3 days of reactivation, total CD4⁺ T cells isolated using Mojosort Kit (Biolegend). 5-10×10⁶ CD4⁺ T cells were transferred intravenously into given genotypes of mice that were sub-lethally irradiated (400 rad) the day before transfer. Recipients also received 80 ng Pertussis toxin intraperitoneally on day 0 and day 1 of transfer. Mice were monitored daily and disease severity was scored as follows: 0=no clinical signs of paralysis, 1=tail paralysis, 2=tail paralysis and hind limb weakness, 3=Complete hind limb weakness, 4=forelimb paralysis or moribund.

Histology

On day 28 after adoptive transfer, mice were sacrificed and perfused with ice cold PBS. Spinal cords were collected and fixed with 10% neutral-buffered formalin for 36 h. Fixed tissues submitted to Cincinnati Children's Hospital Medical Center research pathology core for tissue embedding and Luxol fast blue stain. Slides were imaged at 10× using a Nikon Eclipse Tt.

Quantification and Statistical Analysis

Based on previous and preliminary studies in our lab, we predicted that the reported samples sizes would be sufficient to ensure adequate power. Statistical analyses were performed in Prism (Graph pad) using unpaired or paired Student's t test as indicated in the figure legends. Data are presented as means+/−SEM. Significance was considered at *p<0.05, **p<0.01, ***p<0.001, ****p<0.0001. n.s.=not significant.

Results

T Cell-Interacting BMDCs Produce IL-1β.

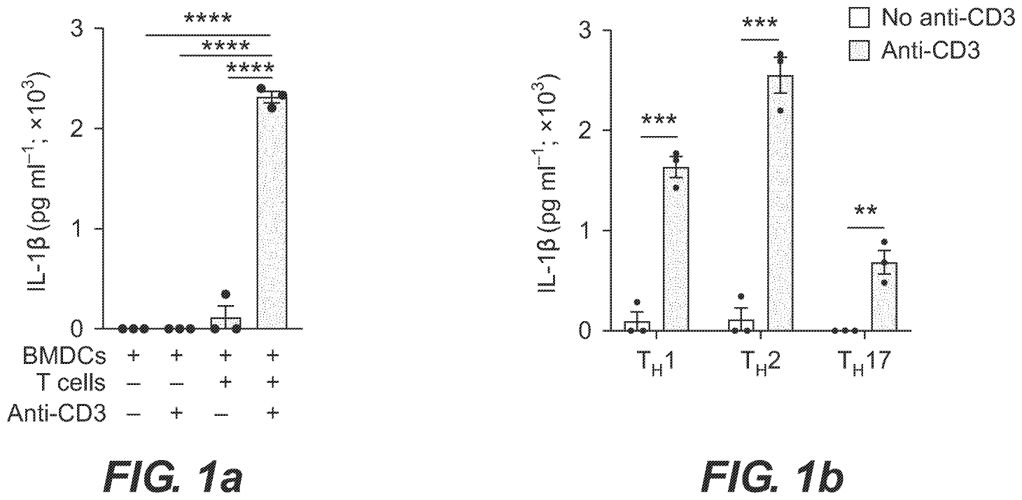

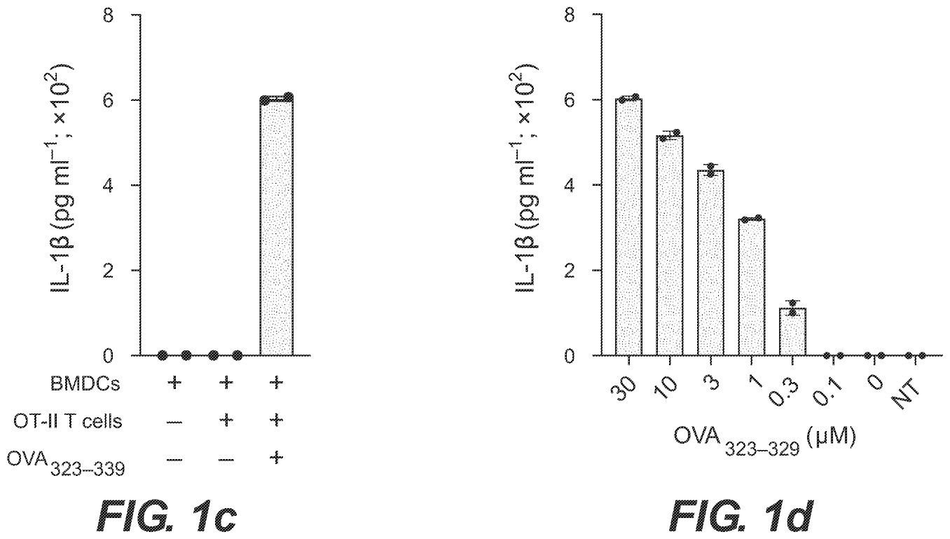

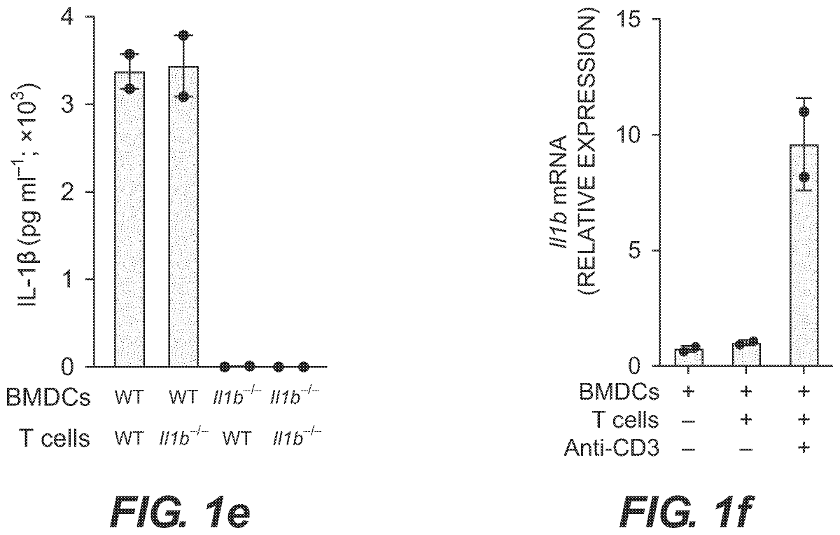

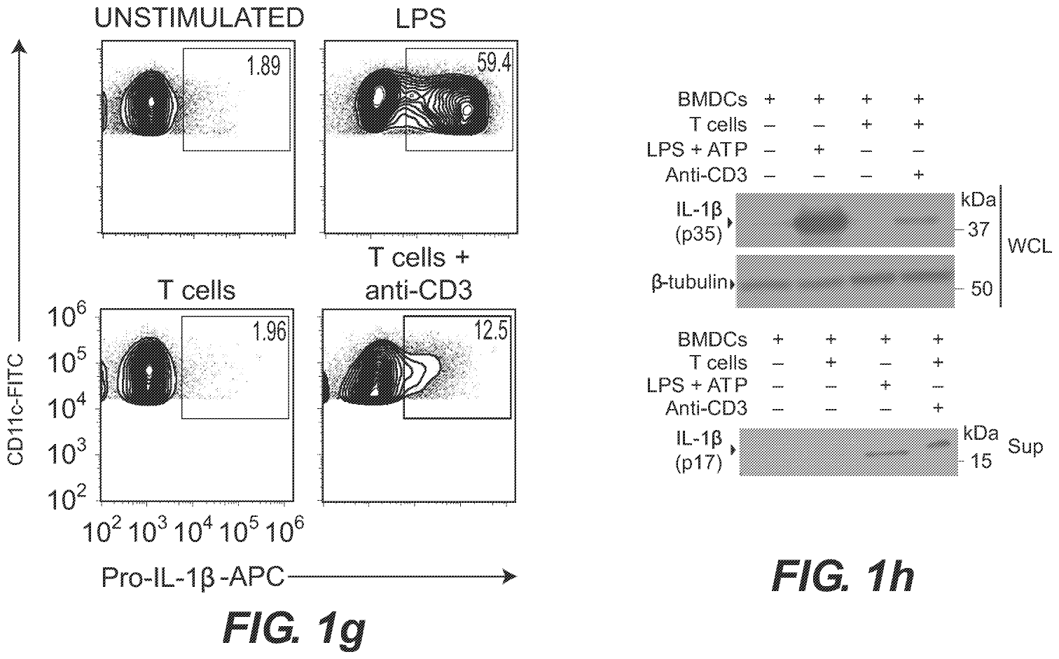

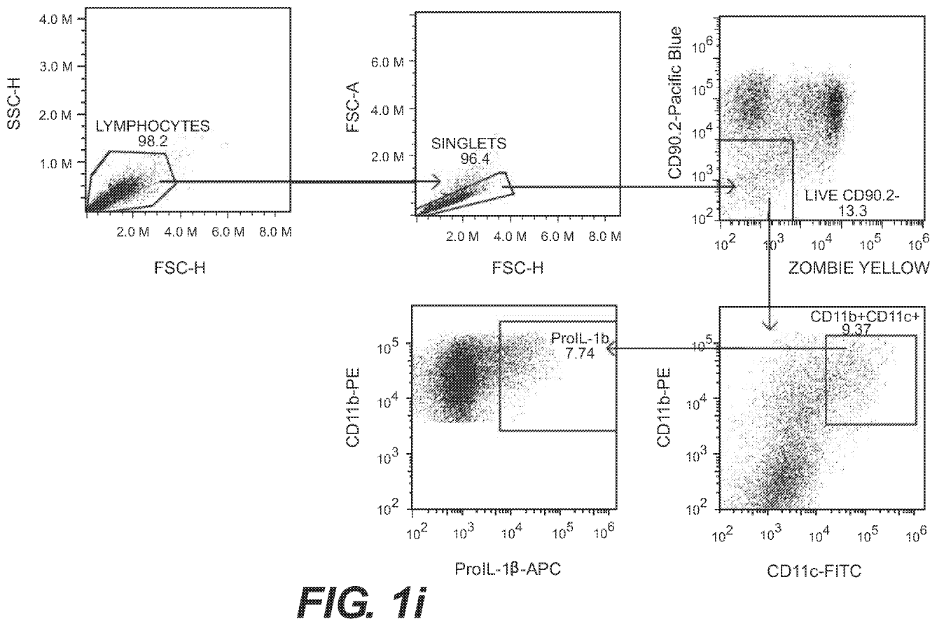

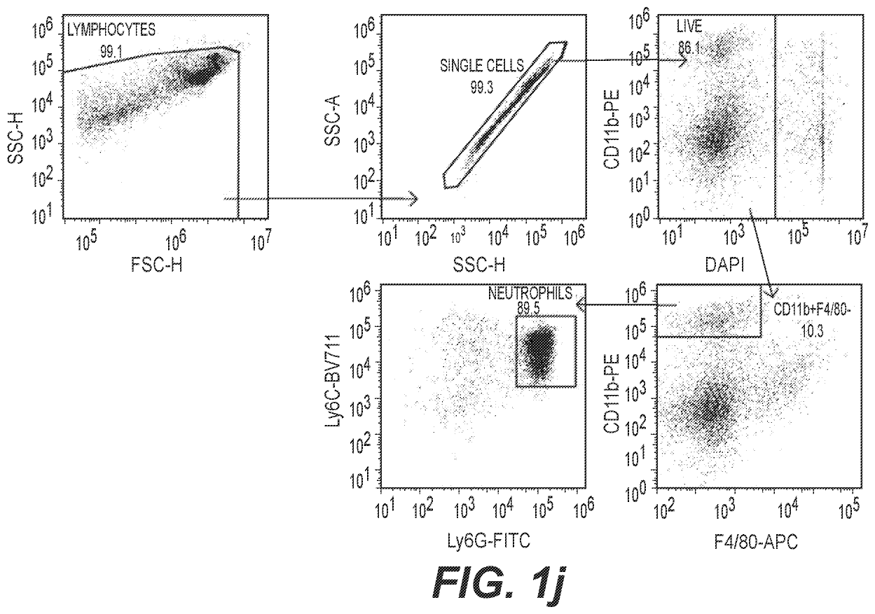

T cell-intrinsic signaling through IL-1R can sometimes be critical for optimal cytokine production by effector and memory CD4⁺ T cells following their reactivation by splenic CD11c⁺ DCs. We therefore tested whether cognate interactions between DCs and effector CD4⁺ T cells could elicit the production of IL-1β independently of signaling through PRRs. We generated bone marrow-derived DCs (BMDCs) derived by culturing wild-type bone marrow cells with GM-CSF-containing media for 5 days, and differentiated naïve wild-type CD4⁺ T cells into T_{H}0 lineage cells through 5 days of culture with plate-bound CD3 Ab and CD28 Ab in the presence of IL-2. BMDCs were co-cultured with rested T_{H}0 CD4⁺ T cells and their interaction was induced with soluble CD3 Ab. In order to prevent consumption of IL-1β by T cells, we added IL-1R antagonist (IL-1Ra), which acts as a potent biological inhibitor of IL-1β. The CD3 Ab-mediated interaction between BMDCs and effector CD4⁺ T cells induced the secretion of IL-1β (FIG. 1a). Furthermore, interaction of BMDCs with CD4⁺ T cells differentiated into T_{H}1, T_{H}2 or T_{H}17 cells using in vitro cytokine polarization also led to secretion of IL-1β (FIG. 1b), indicating a broadly-conserved pathway of IL-1β production across multiple T cell lineages. Because all CD4⁺ T cell lineages instructed the production of IL-1β in BMDCs, we used differentiated CD4⁺ T_{H}0 cells, hereafter referred to as effector CD4⁺ T cells unless otherwise noted, for all further experiments. Next, we examined if production of IL-1β was dependent on the presentation of cognate peptide by BMDCs. In co-cultures of OVA peptide-restricted OT-II TCR transgenic effector CD4⁺ T cells and wild-type BMDCs, production of IL-1β was detected in the presence of the cognate OVA₃₂₃₋₃₃₉ peptide (FIG. 1c). Additionally, the amount of IL-1β detected in the supernatants directly correlated with the concentration of OVA₃₂₃₋₃₃₉ provided in the cultures (FIG. 1d), indicating that the avidity of the MHC-TCR interaction was a major determinant of the quantity of IL-1β secreted. CD4⁺ T cells have been reported to produce IL-1β. However, we found that use of Il1b^{-/-} BMDCs, but not Il1b^{-/-} T cells, led to a complete loss of T cell-instructed IL-1β production (FIG. 1e), suggesting that BMDCs were the source of IL-1β in this co-culture system. Co-culture of Il1b^{-/-} CD4⁺ T cells with wild-type BMDCs induced the Il1b transcript 3 h post-stimulation (FIG. 10, and intracellular pro-IL-1β could be detected by flow cytometry in CD11c⁺ BMDCs 6 h post-stimulation (FIG. 1g and FIG. 1i). Total and cleaved bioactive IL-1β were detected by immunoblotting analysis of the co-culture whole cell lysate and supernatant, respectively, after 18 h of stimulation (FIG. 1h). These data indicated that the interaction between BMDCs and effector CD4⁺ T cells triggered the transcriptional induction of pro-IL-1β and its bioactive cleavage in BMDCs.

TNFR Signaling in BMDCs Leads to Pro-IL-1β Synthesis

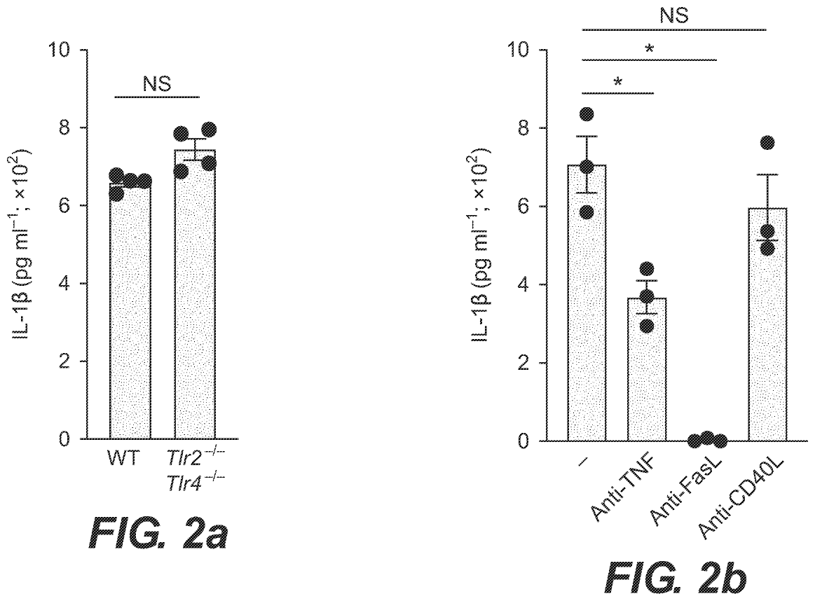

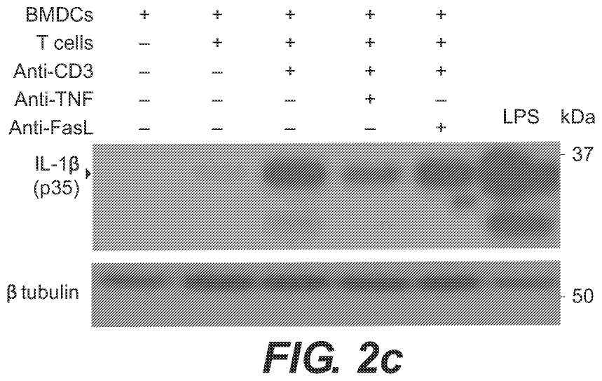

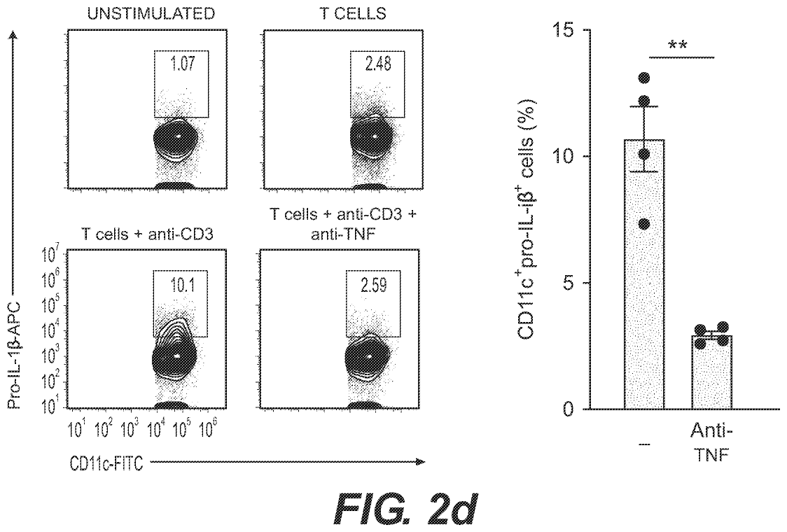

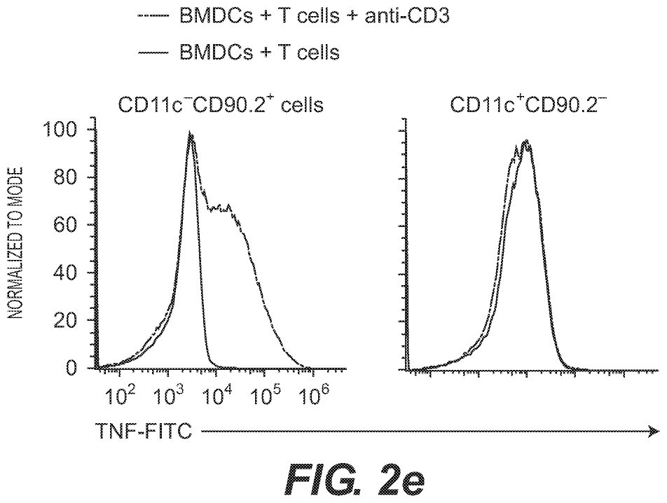

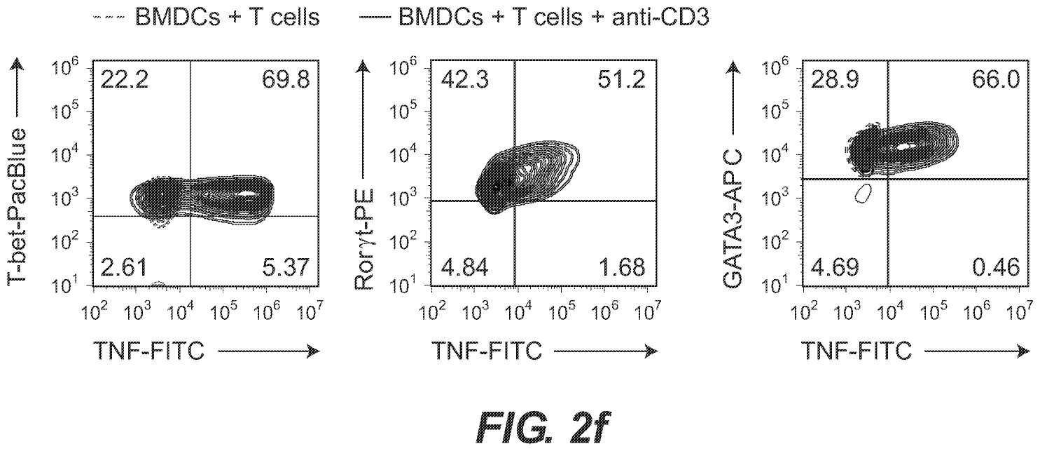

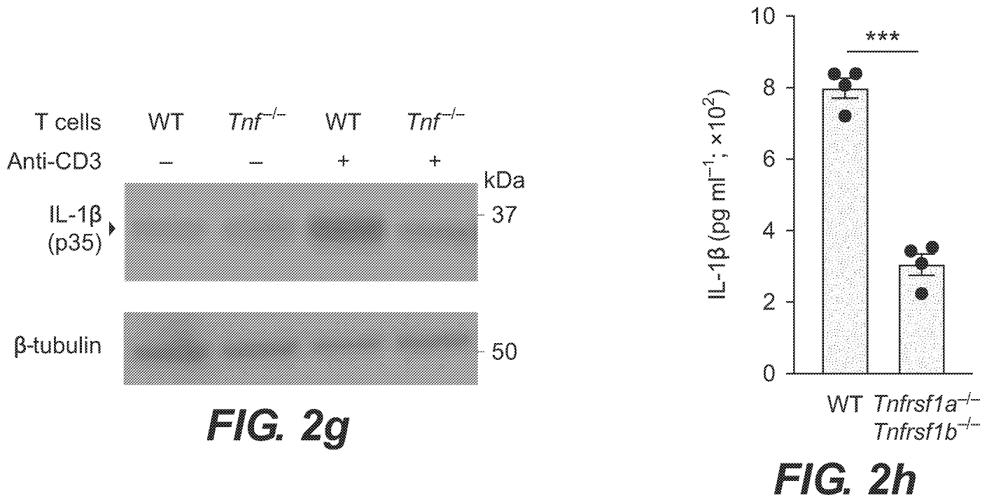

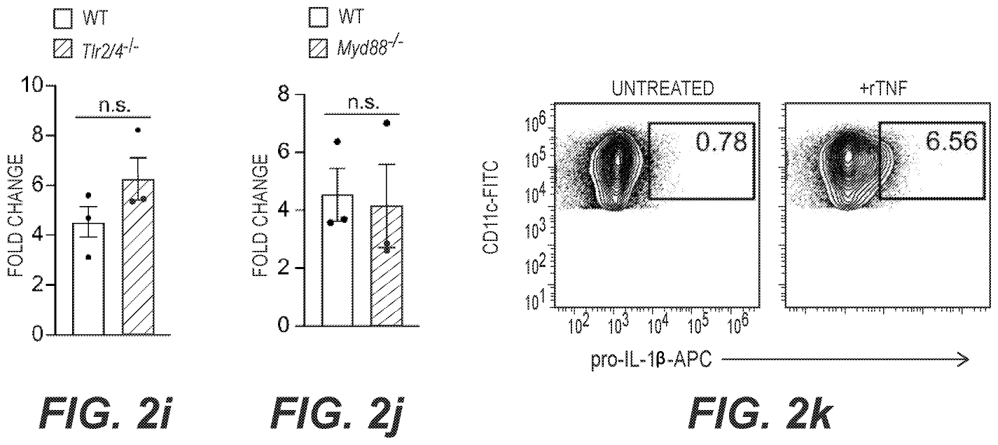

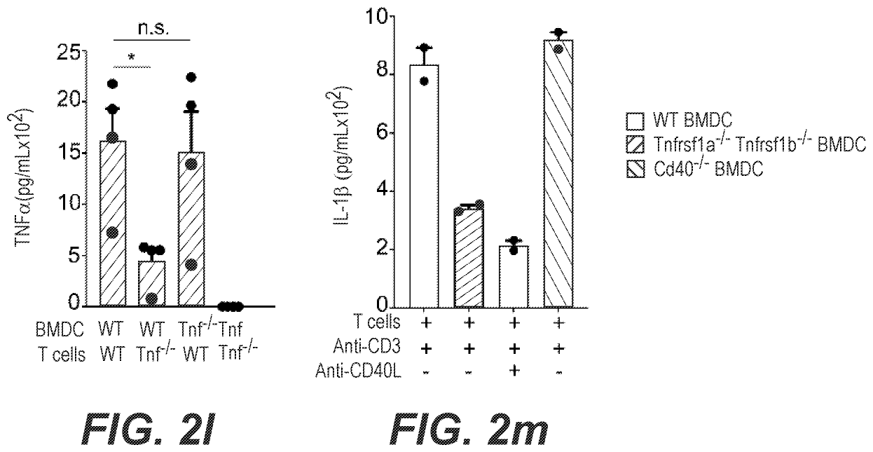

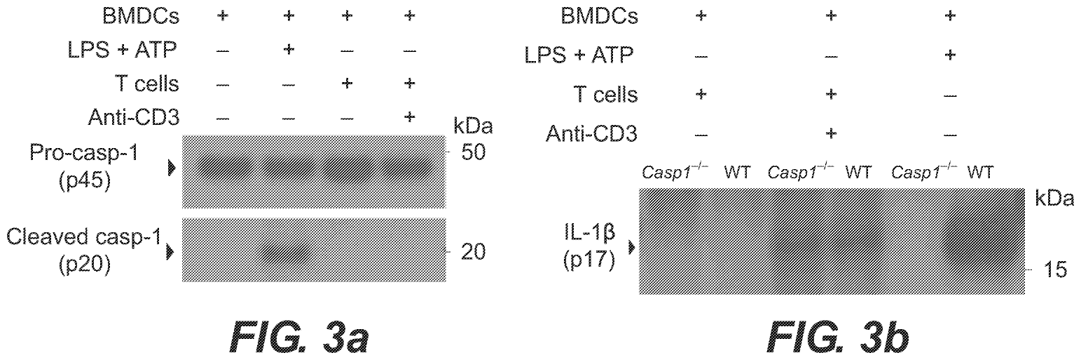

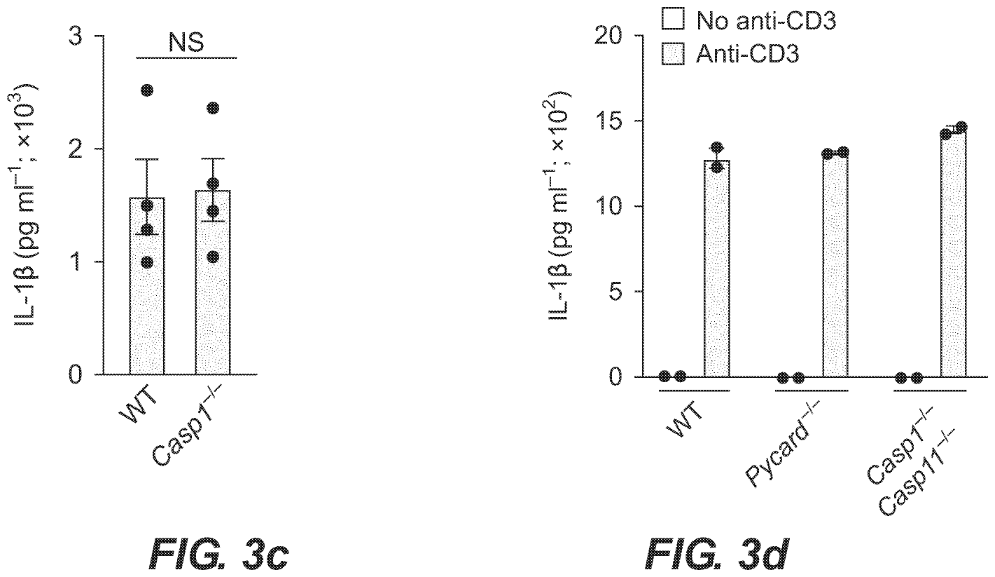

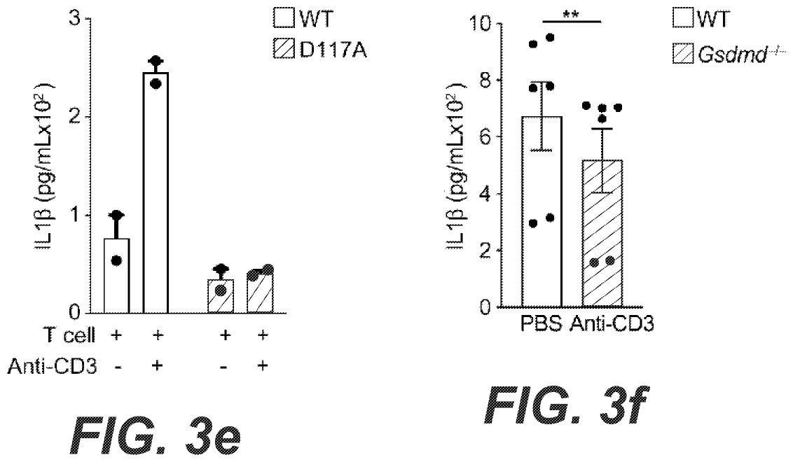

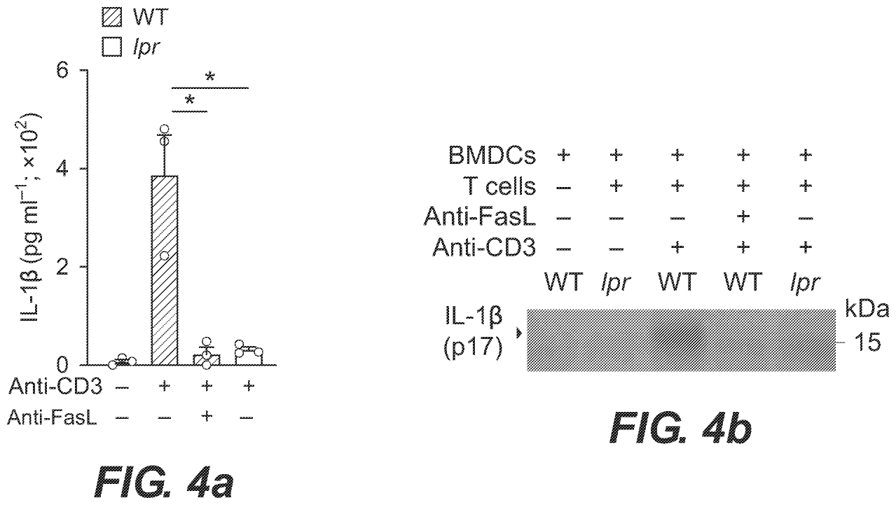

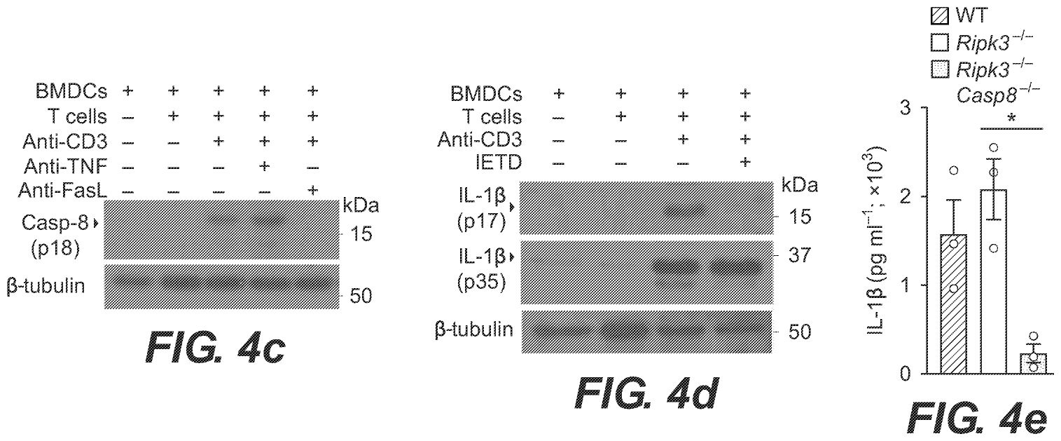

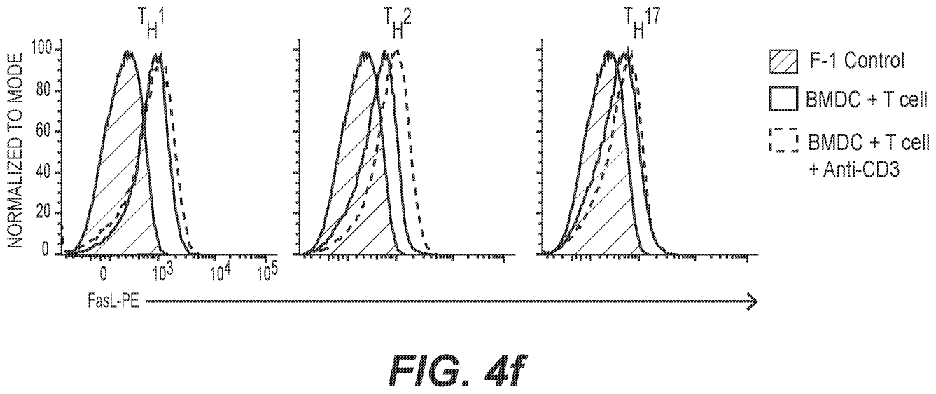

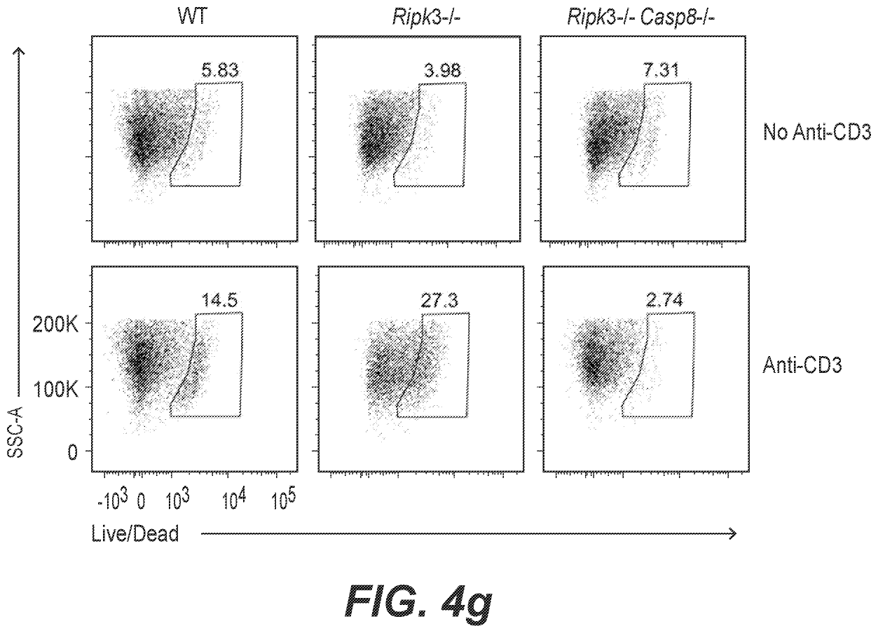

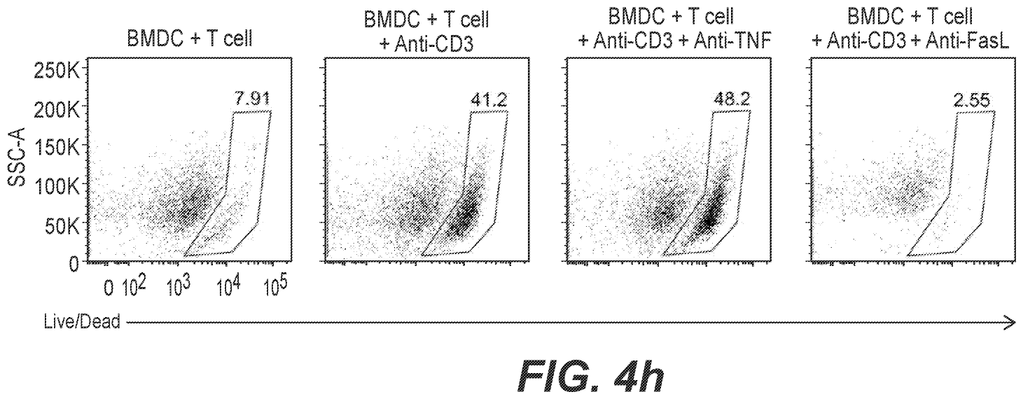

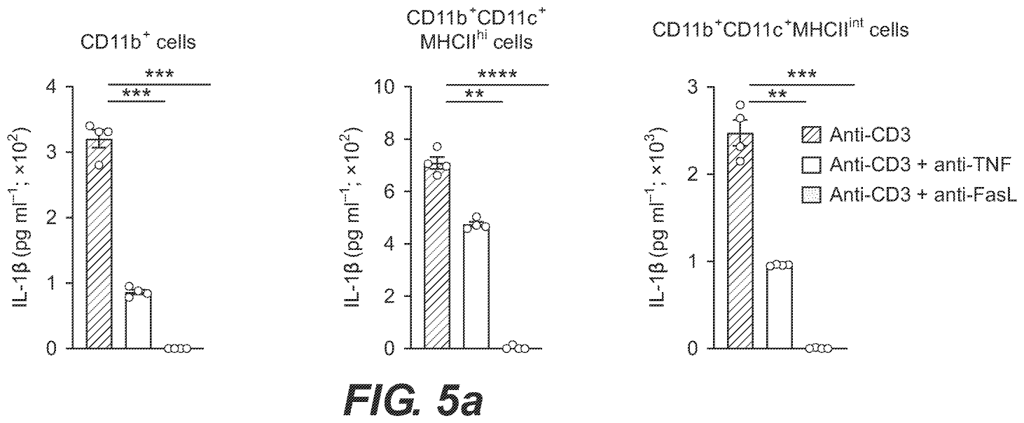

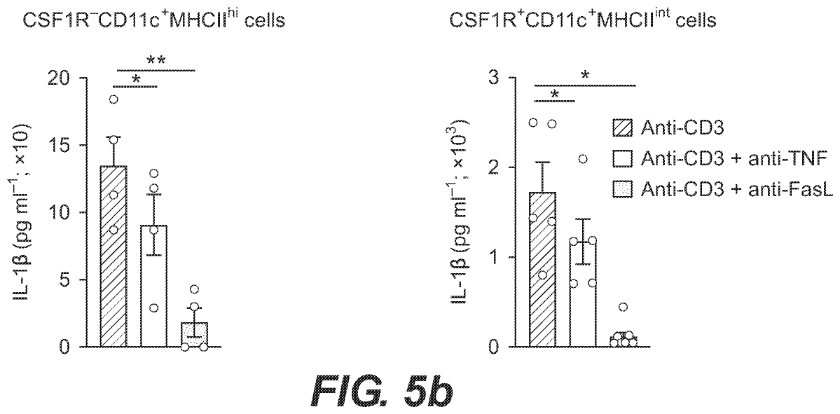

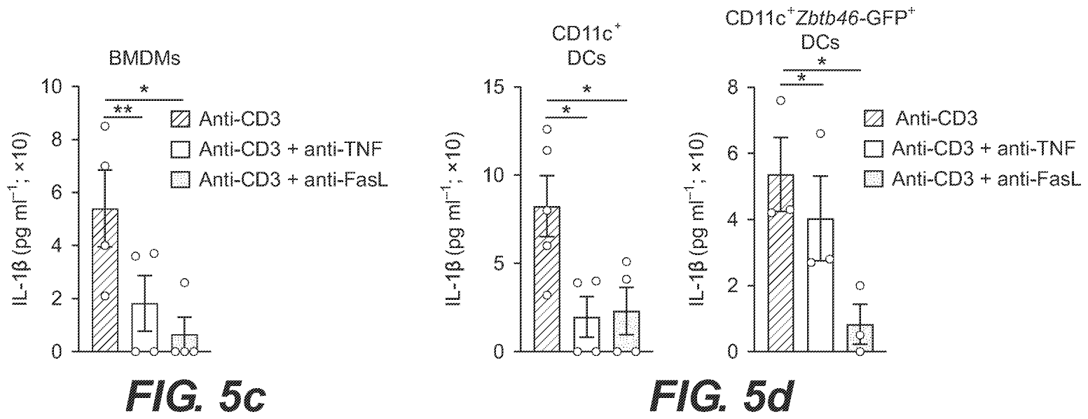

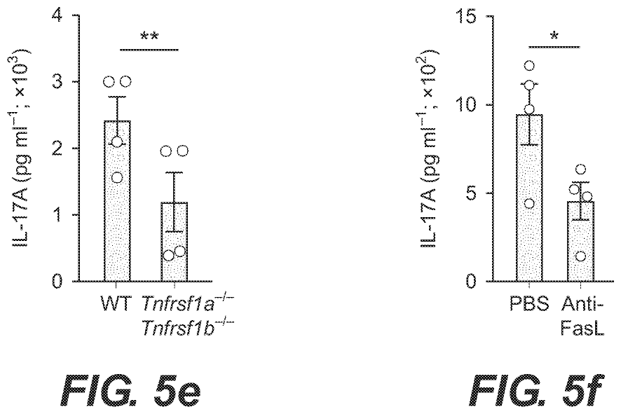

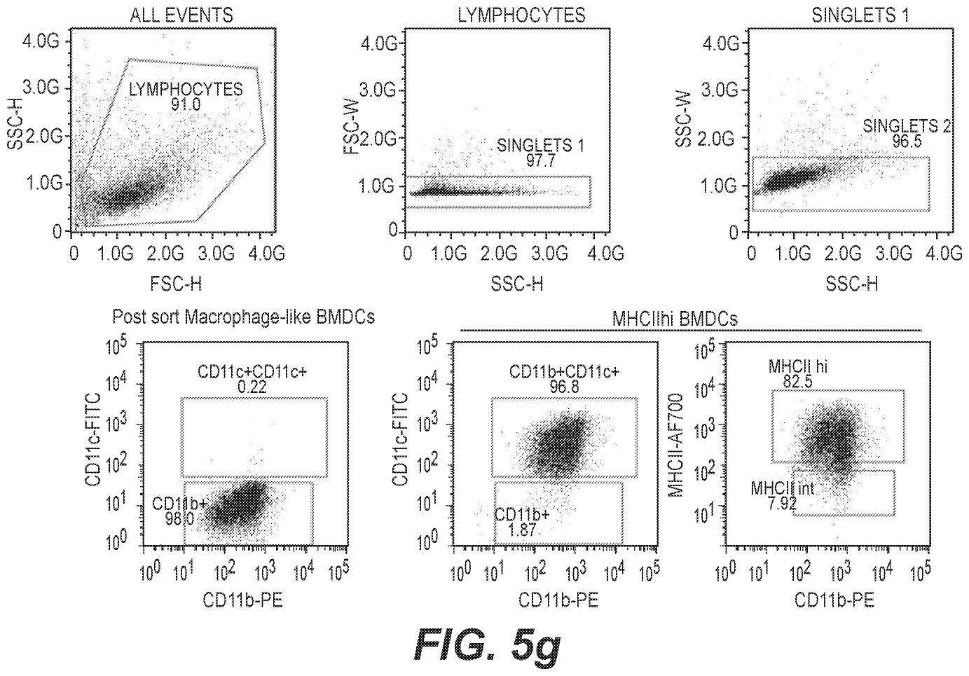

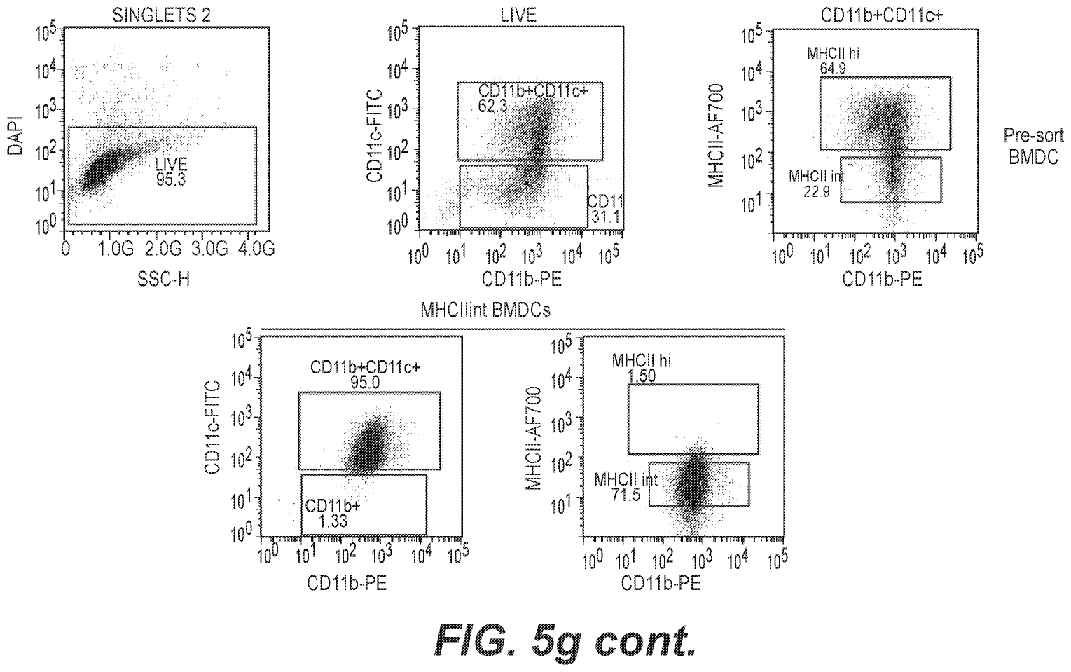

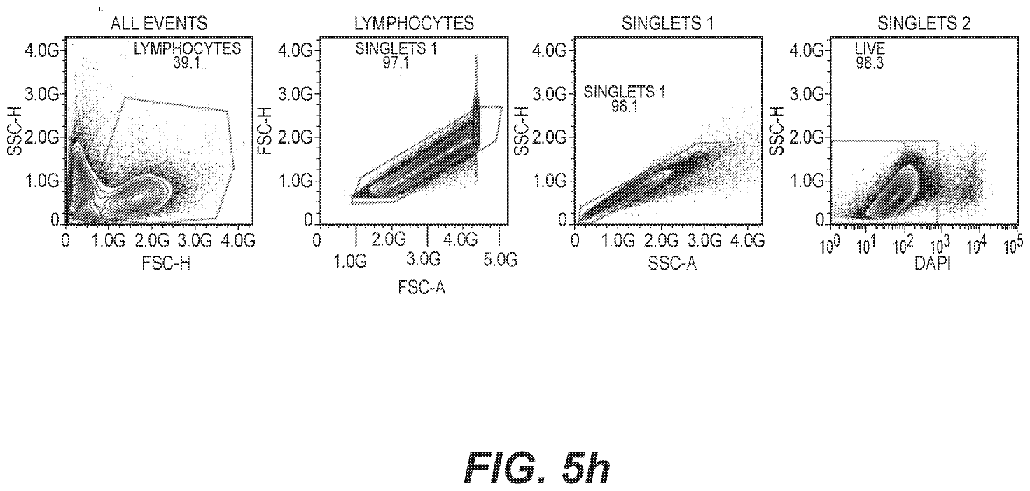

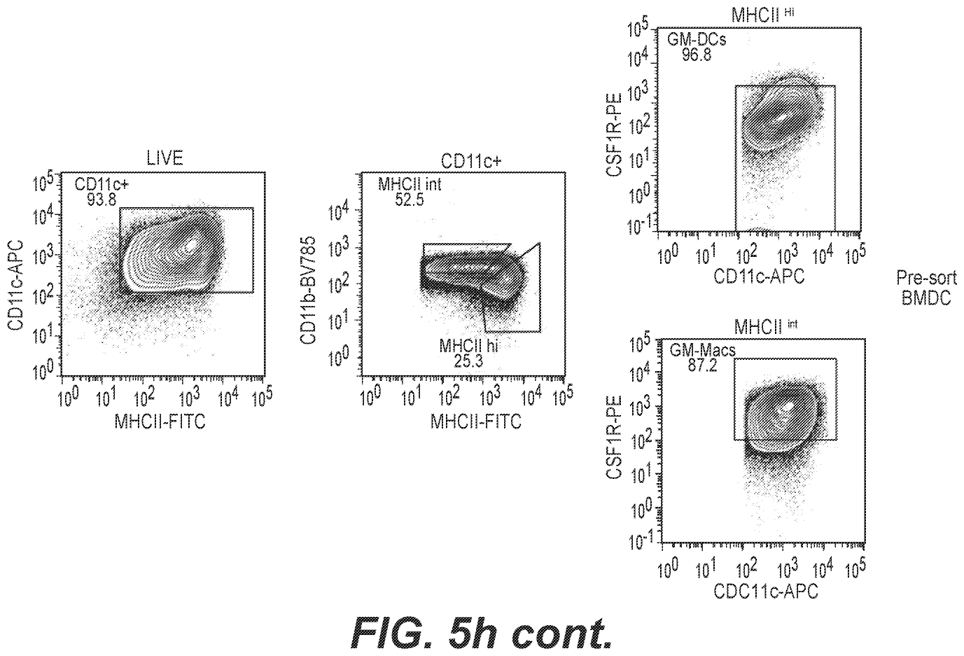

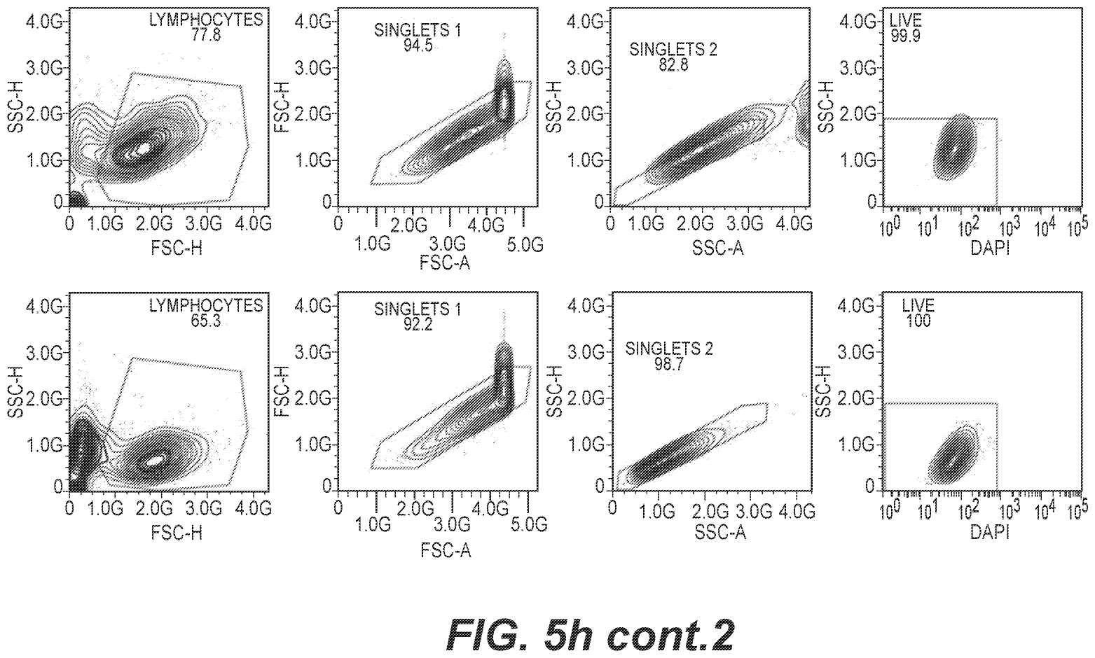

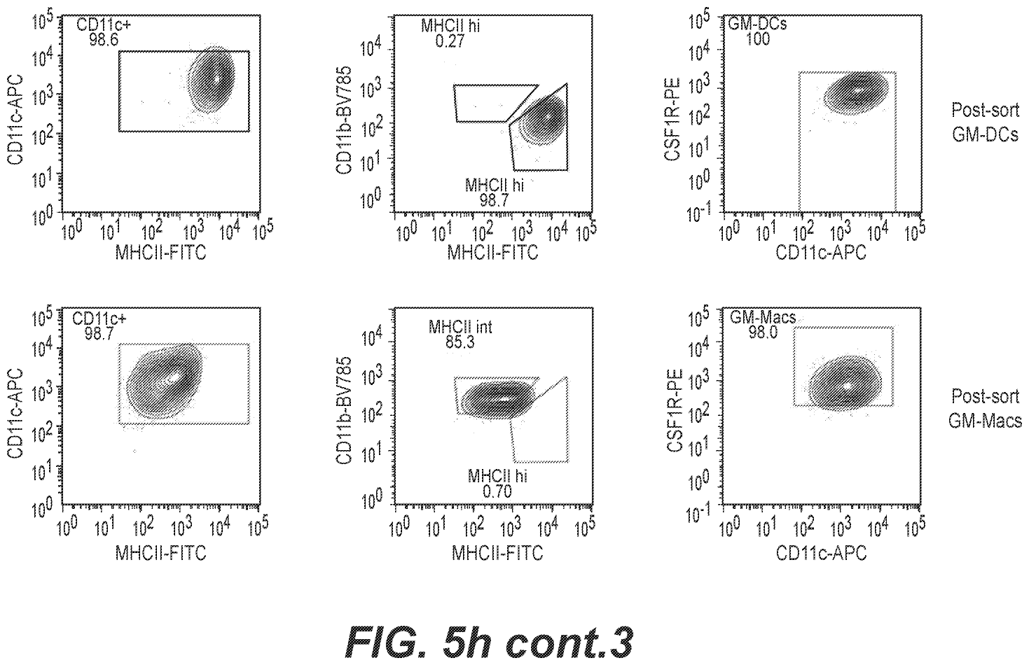

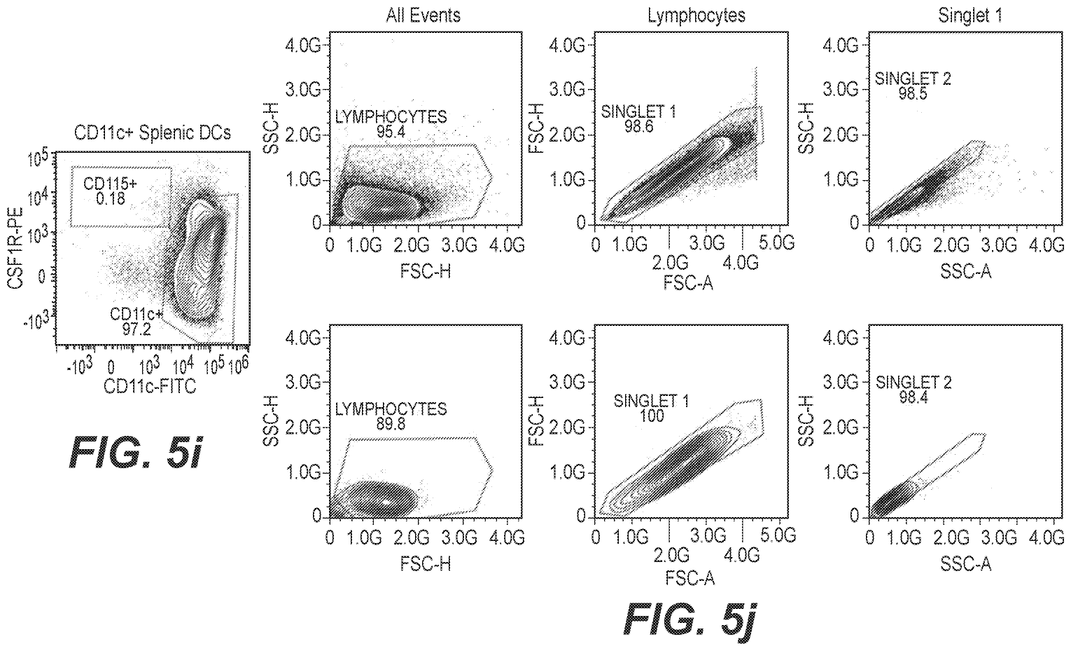

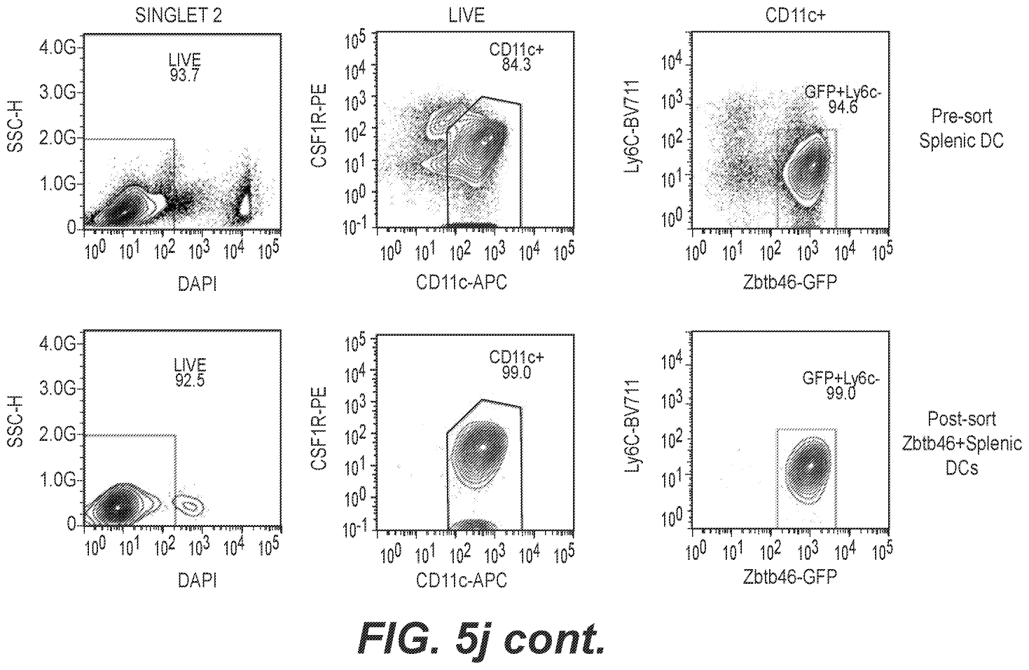

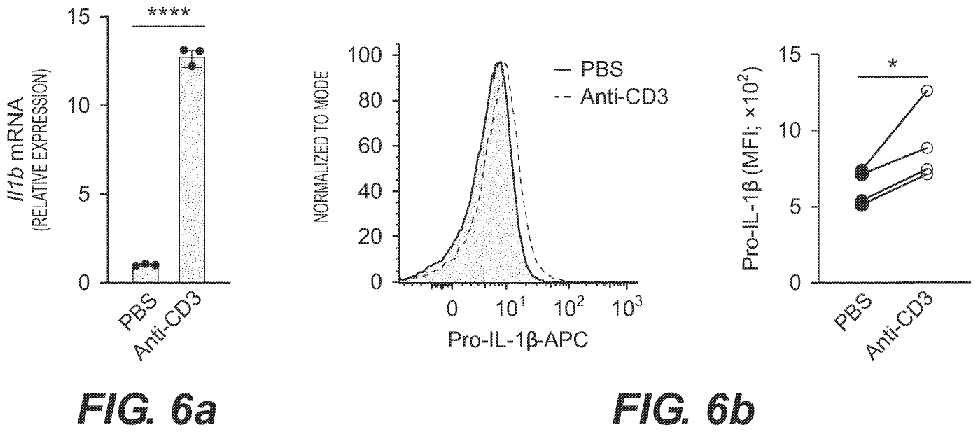

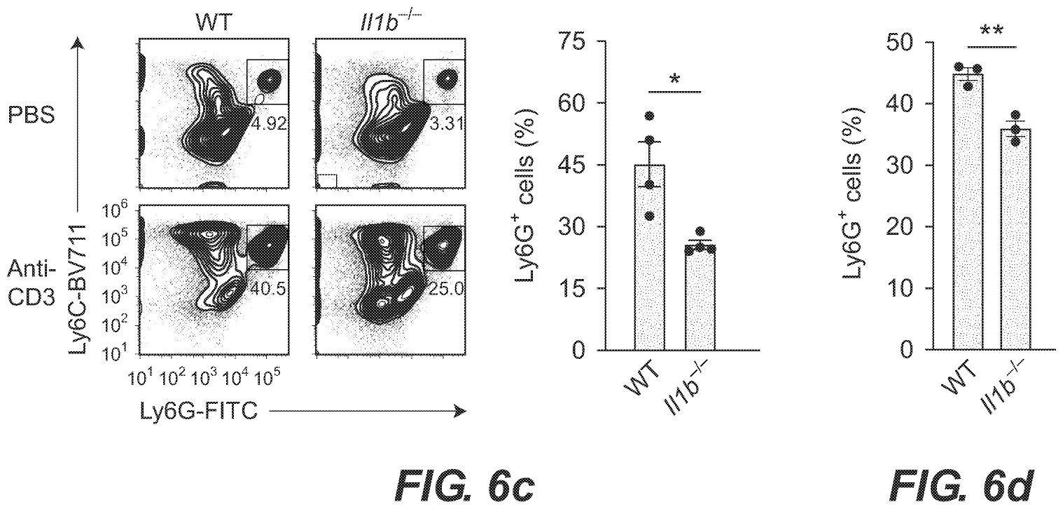

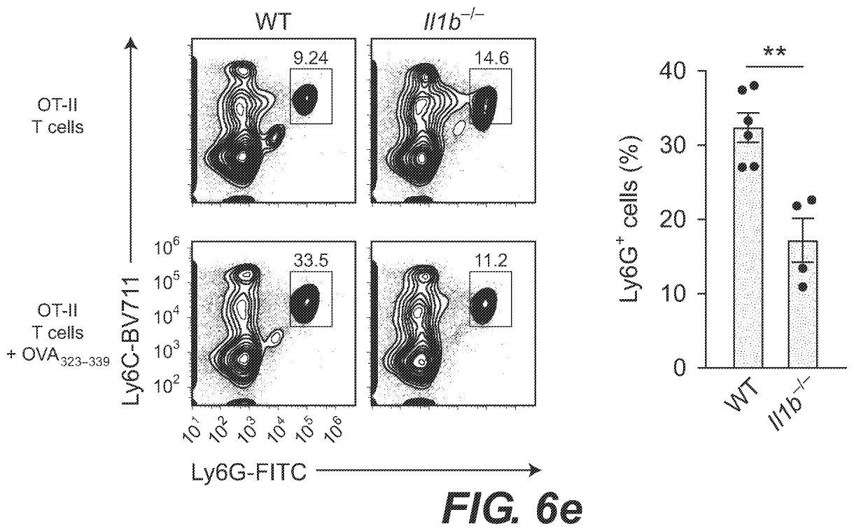

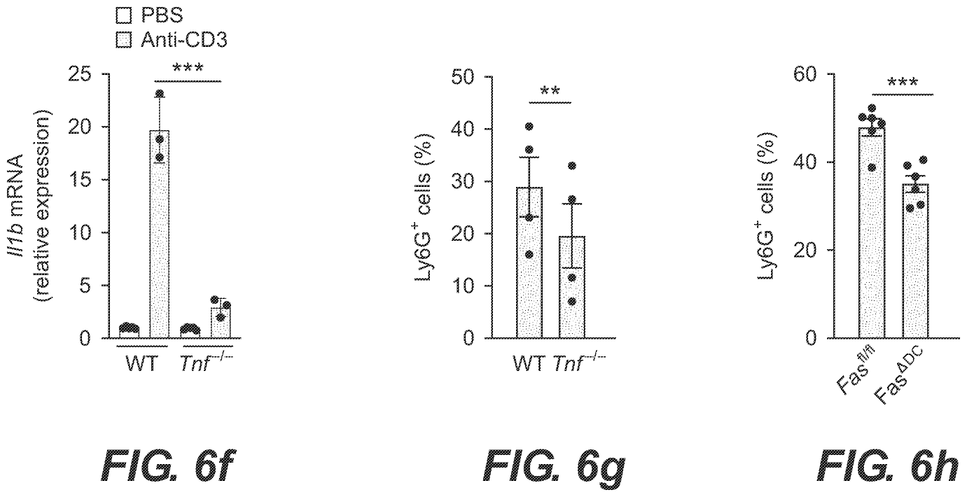

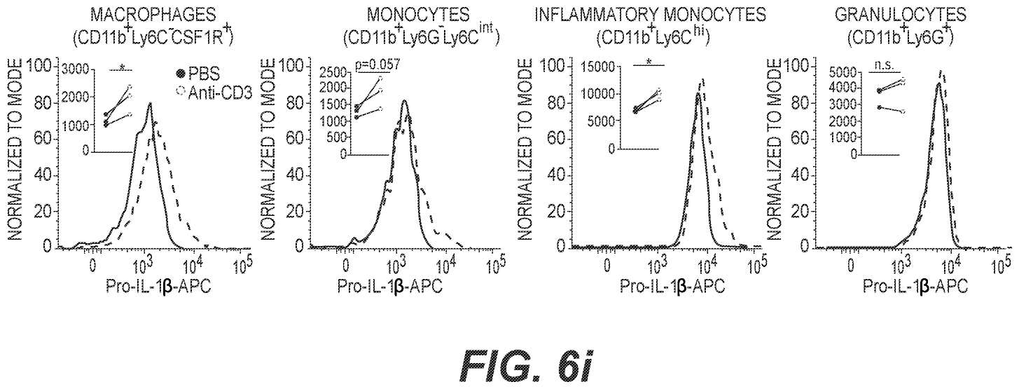

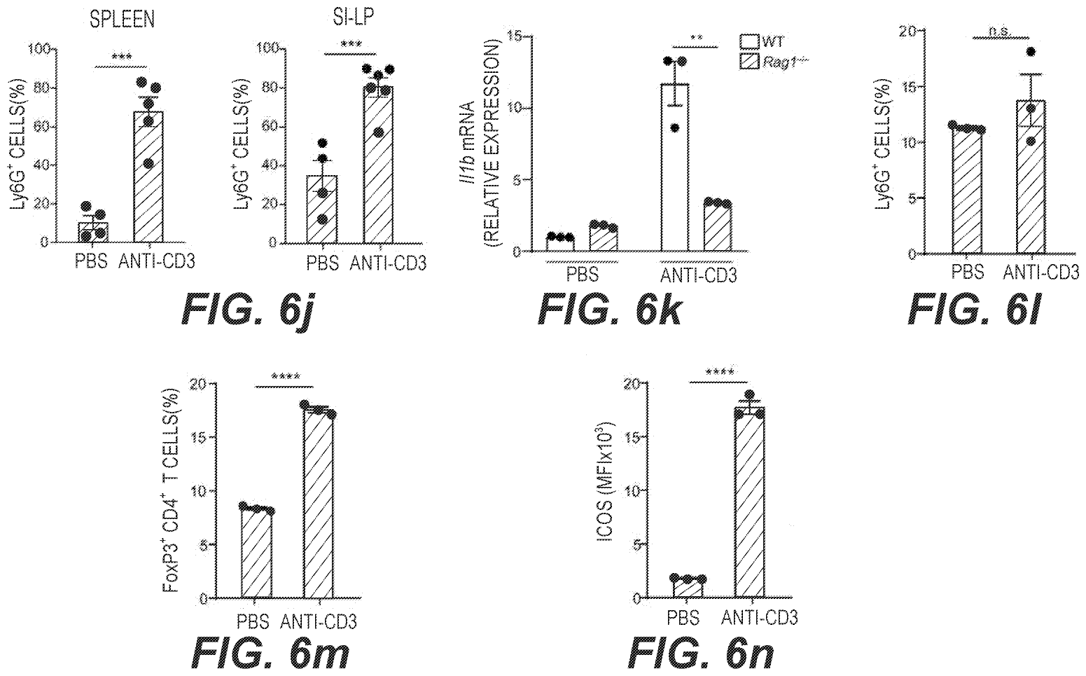

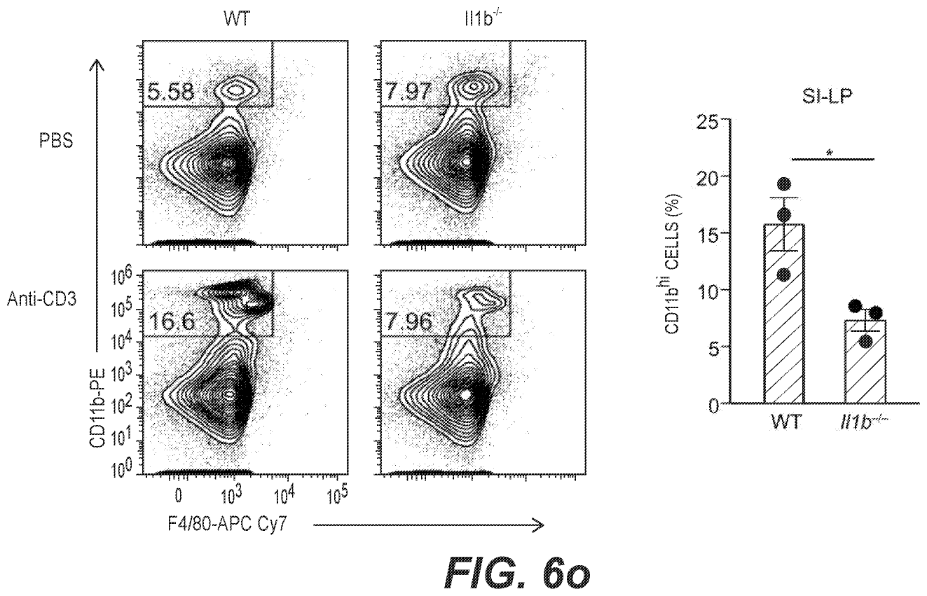

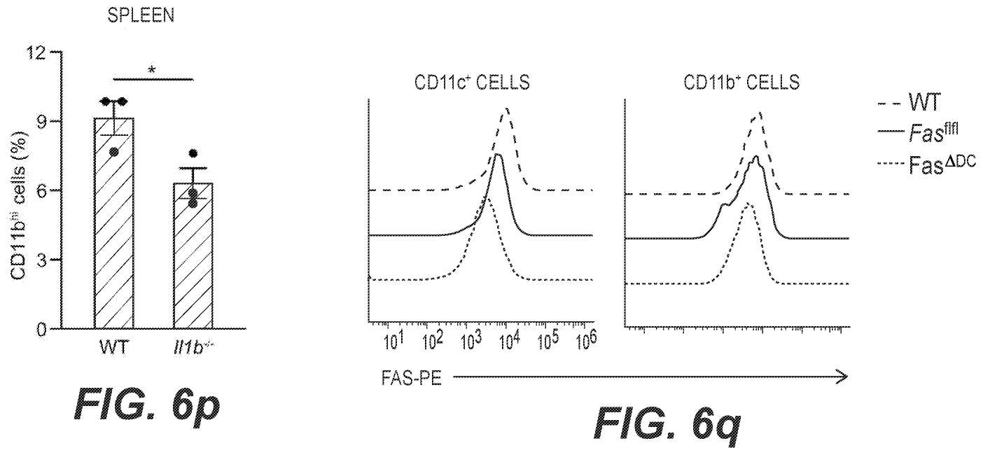

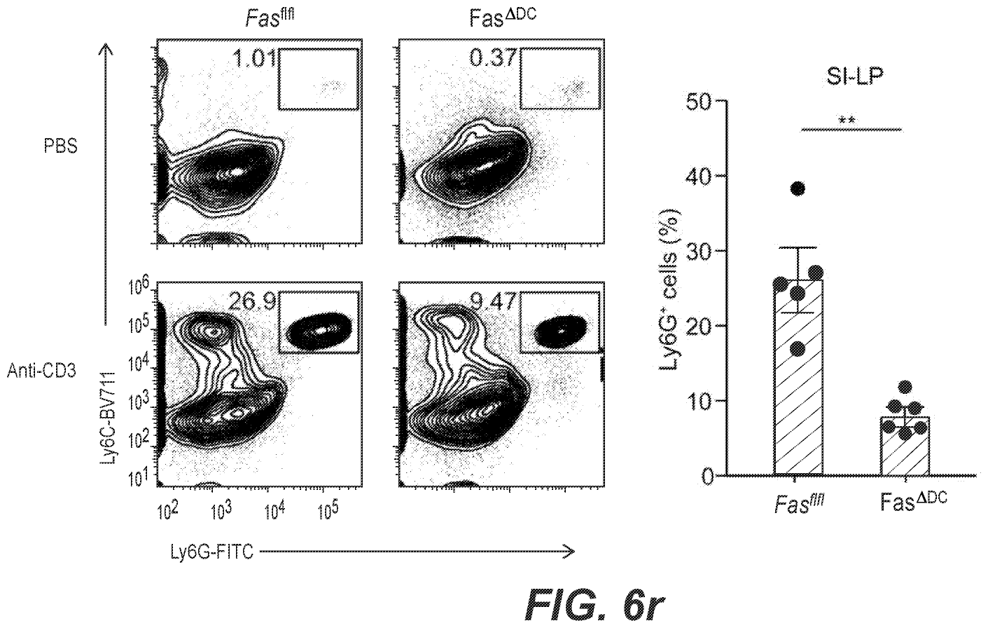

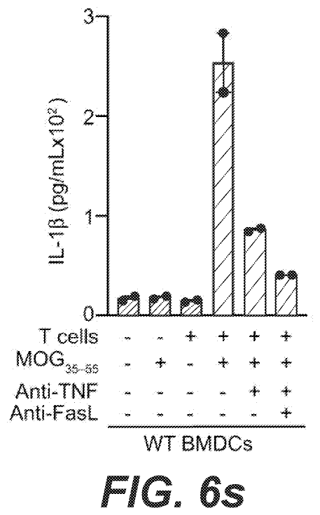

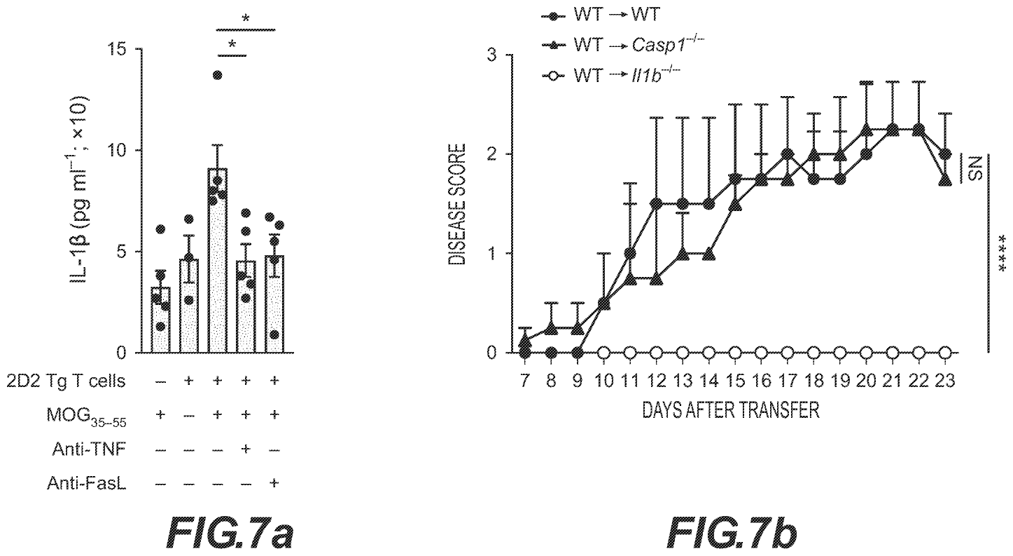

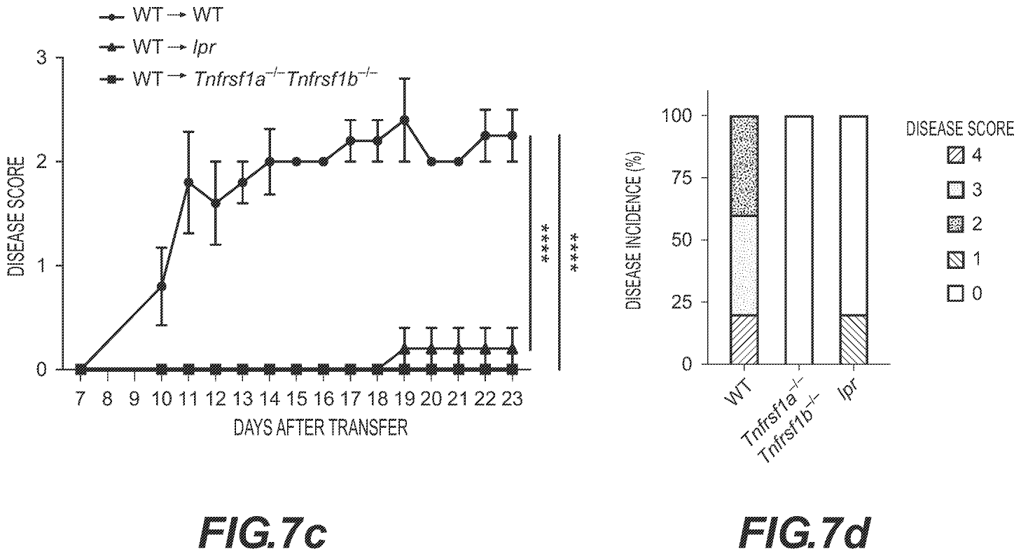

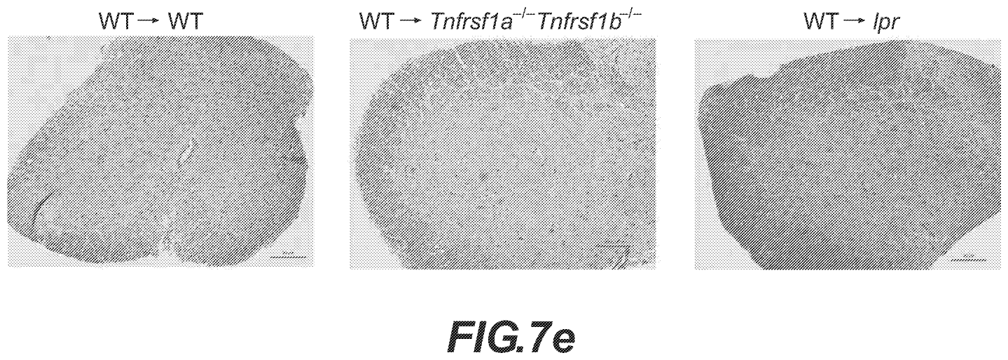

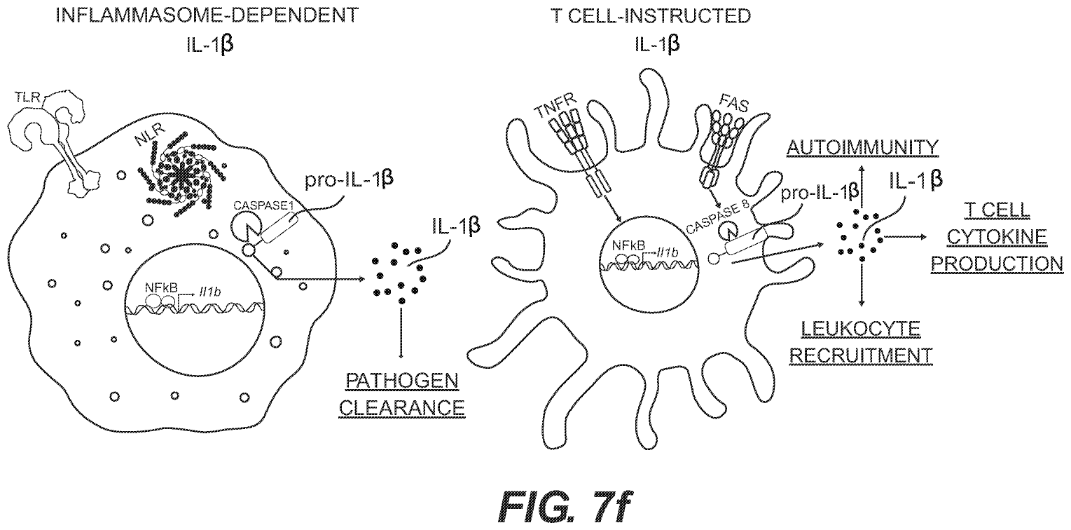

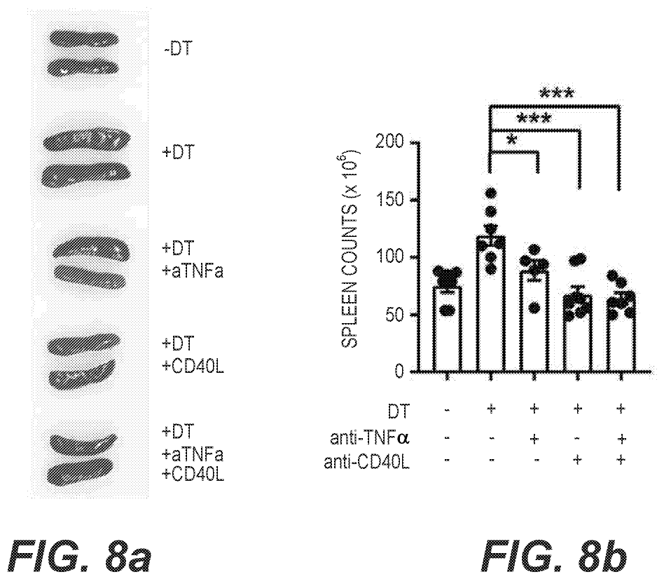

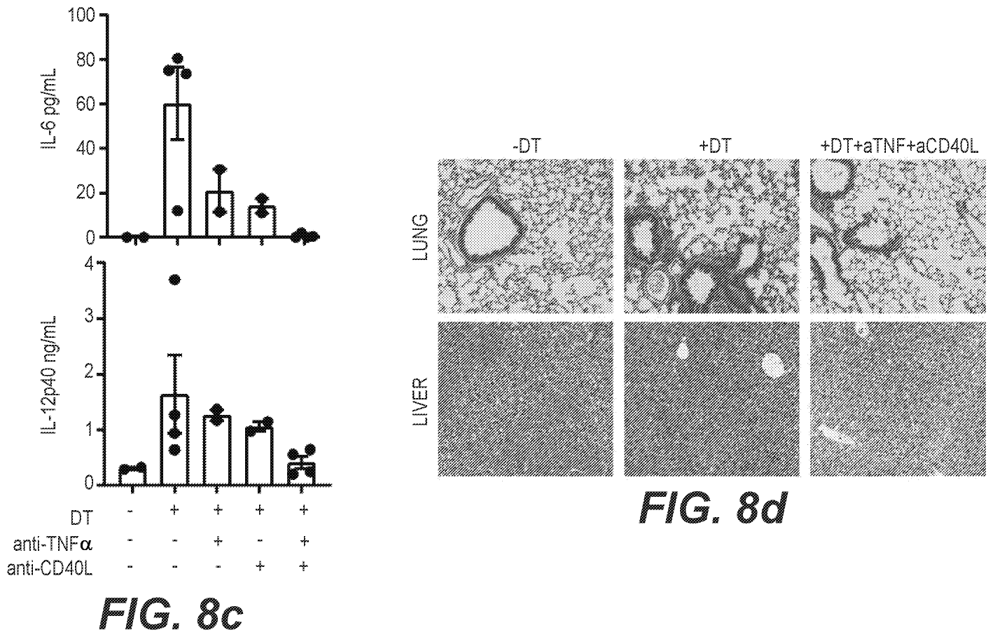

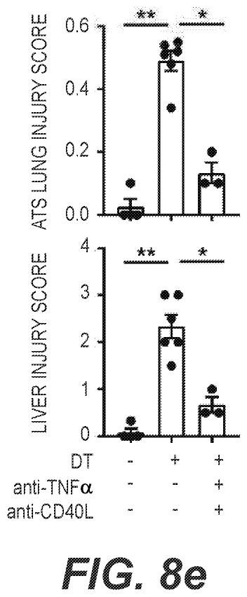

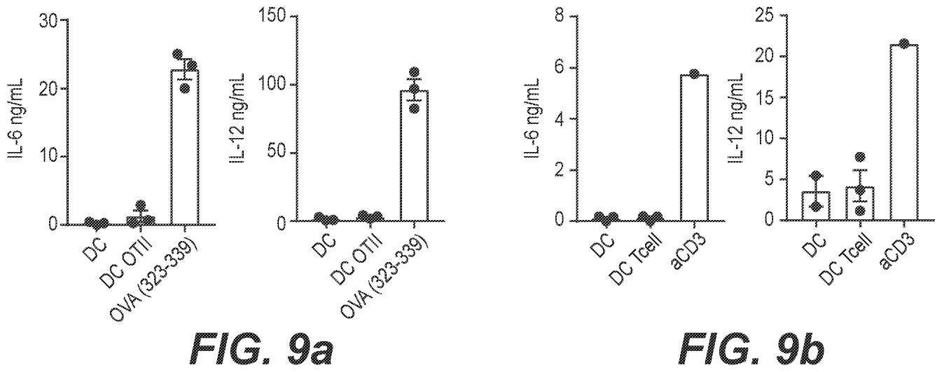

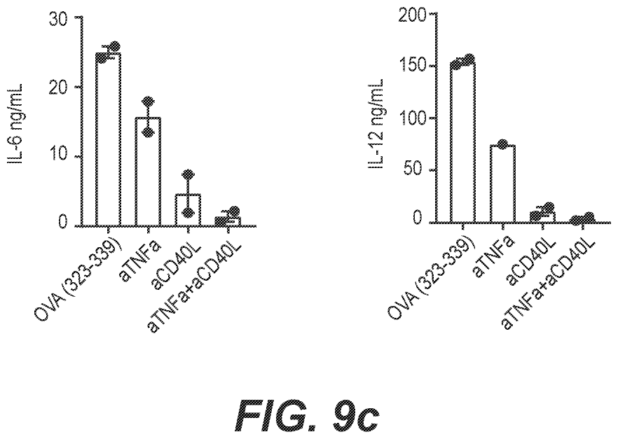

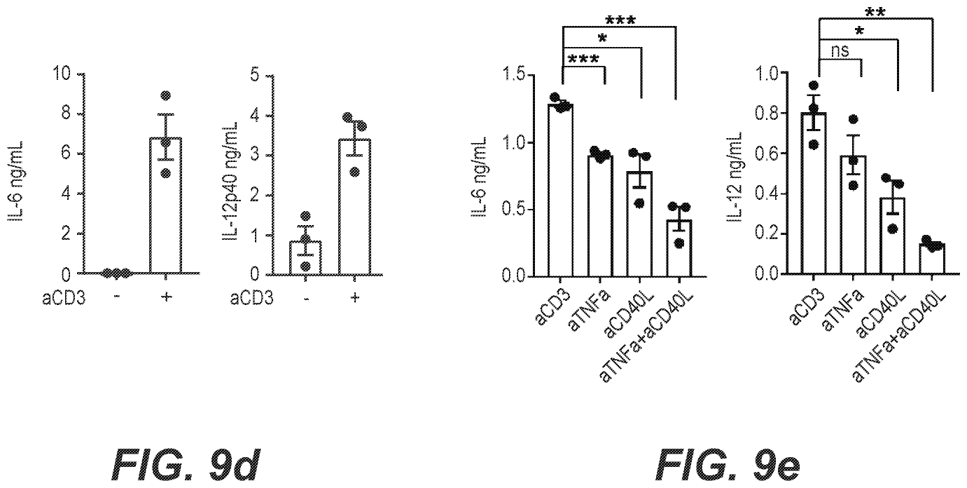

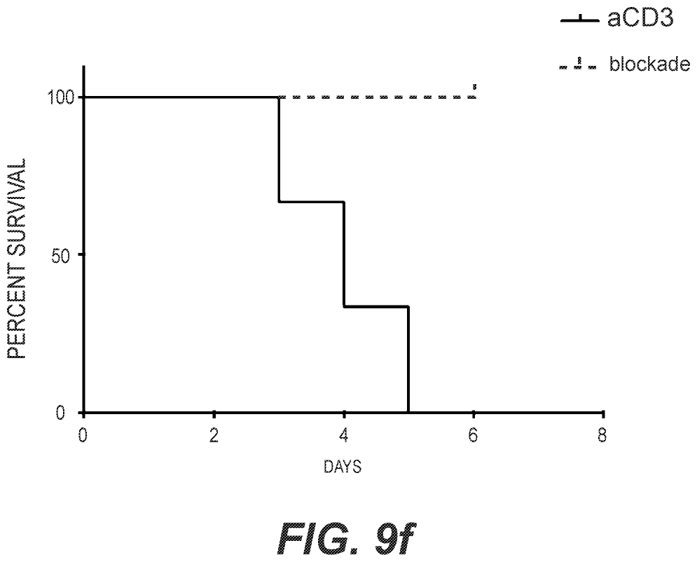

Next, we characterized the signaling events which enabled the BMDC intrinsic production of T cell-induced IL-1β. Inflammasome activation is a mechanism for the production of IL-1β, in which TLR signaling is responsible for the transcriptional upregulation of pro-IL-1β. Induction of intracellular pro-IL-1β and secretion of mature IL-1β in Tlr2^{-/-}Tlr4^{-/-} or Myd88^{-/-} BMDCs co-cultured with effector CD4⁺ T cells, however, was similar to that of wild-type BMDCs (FIGS. 2a, 2i, and 2j), ruling out endotoxin contamination in these cultures. Because transcription of pro-IL-1β is mediated by NF-κB and AP-1, which can be activated downstream of TNF superfamily receptors, we examined the role of TNF receptors in the T cell-induced production of pro-IL-1β in BMDCs. Antibody-mediated neutralization of TNF and FasL, but not CD40L, reduced the production of IL-1β compared to PBS (FIG. 2b). Pro-IL-1β was reduced by TNF, but not FasL, neutralization as assessed by immunoblotting (FIG. 2c), suggesting that Fas-FasL interaction could sometimes be critical for the production of mature IL-1β, but not the transcriptional induction of pro-IL-1β. Consistent with this finding, neutralization of TNF in wild-type BMDCs-effector CD4⁺ T cell co-cultures reduced the amount of intracellular pro-IL-1β detected by flow cytometry, as compared to PBS (FIG. 2d). Moreover, stimulation with recombinant TNF drove the synthesis of pro-IL-1β in wild-type BMDCs (FIG. 2k).

Although TNF is largely of myeloid origin during PRR-driven inflammation, we found that reactivating Tnf effector CD4⁺ T cells with wild-type BMDCs resulted in less secreted TNF compared to wild-type effector CD4⁺ T cells co-cultured with wild-type BMDCs (FIG. 2e and FIG. 2l), indicating that activated T cells were the predominant producers of TNF during interaction with BMDCs. While TNF is primarily known to be an effector cytokine for T_{H}17 cells, primed T_{H}1 and T_{H}2 cells also rapidly upregulated TNF upon interaction with BMDCs (FIG. 2f), suggesting that multiple activated CD4⁺ T cell lineages can signal through the TNFR on BMDCs. Indeed Tnf^{-/-} effector CD4⁺ T cells induced appreciably diminished pro-IL-1β in wild-type BMDCs compared to wild-type effector CD4⁺ T cells (FIG. 2g). Moreover, Tnfrsf1a^{-/-} Tnfrsf1b^{-/-} BMDCs secreted less T cell-induced IL-1β compared to wild-type BMDCs (FIG. 2h), indicating that TNFR signaling in BMDCs appeared to be required for optimal production of IL-1β. Because IL-1β production was not completely abrogated in Tnfrsf1a^{-/-}Tnfrsf1b^{-/-} BMDCs, we examined if there was also a role for CD40 signaling in this context. Blocking CD40L using neutralizing antibody in Tnfrsf1a^{-/-}Tnfrsf1b^{-/-} BMDCs further reduced the production of IL-1β (FIG. 2m). These data indicate that TNFR signaling can lead to induction of IL-1β in BMDCs during their interaction with CD4⁺T cells, while other TNF superfamily proteins, such as CD40, could contribute to this induction in the absence of TNFR signaling.

T Cell-Instructed IL-1β is Independent of Casp-1

Casp-1, the effector protease of inflammasomes, is largely responsible for the production of bioactive IL-1β through its cleavage of pro-IL-1β at the aspartate residue in position 117 (D117). Expression of an D117A IL-1β mutant in Il1b^{-/-} BMDCs co-cultured with effector CD4⁺ T cells led to less secretion of IL-1β compared to expression of wild-type IL-1β (FIG. 3e), indicating the D117 casp-1 cleavage site of pro-IL-1β could sometimes be critical for the T cell-induced production of IL-1β in BMDCs. Despite this, we did not detect active casp-1 in T cell-interacting BMDCs after 18 h of co-culture (FIG. 3a). Furthermore, Casp1^{-/-} BMDCs showed no reduction in T cell-induced IL-1β production compared to wild-type BMDCs (FIG. 3b, c). Pycard^{-/-} and Casp4^{-/-} BMDCs, lacking ASC and casp-11 respectively, also had normal production of IL-1β (FIG. 3d), indicating T cell-induced IL-1β production was independent of canonical as well as non-canonical inflammasomes. During inflammasome activation, gasdermin-D drives pyroptosis and the secretion of mature IL-1β. Gsdmd−/− BMDCs showed a partial reduction in T cell-induced IL-1β compared to wild-type BMDCs (FIG. 3f). These observations indicate that T cell-induced IL-1β in BMDCs was independent of casp-1 and casp-11, and likely to be entirely independent of inflammasome activation.

Fas-Casp-8 Mediates Maturation of T Cell-Induced IL-1β

Effector CD4⁺ T cells constitutively expressed FasL, which was further upregulated upon CD3 Ab mediated interaction with BMDCs (FIG. 4f). As such, we investigated whether Fas signaling induced cleavage of pro-IL-1β in BMDCs. BMDCs derived from lpr mice, which encodes a spontaneous homozygous loss-of-function mutation in the Fas gene, did not secrete cleaved IL-1β upon interaction with wild-type effector CD4⁺ T cells (FIG. 4a,b), indicating that Fas signaling may be required for production of T cell-induced IL-1β in BMDCs. Fas signaling has been reported to trigger casp-8-dependent cleavage of pro-IL-1β in macrophages. Interaction of BMDCs with effector CD4⁺ T cells led to maturational cleavage of casp-8 in the co-culture whole cell lysate (FIG. 4c). TNFR signaling has been reported to cause casp-8 activation. However, antibody mediated neutralization of FasL, but not TNF, during interaction of BMDCs with effector CD4⁺ T cells led to reduced amounts of cleaved casp-8 in the co-culture whole cell lysate (FIG. 4c). Addition of the casp-8 inhibitor IETD in the BMDCs-effector CD4⁺ T cells co-cultures led to loss of mature IL-1β in the supernatant (FIG. 4d), indicating that casp-8 was the effector protease involved in the maturation of T cell-induced pro-IL-1β in BMDCs. Casp-8 has been reported to induce NF-κB-dependent genes, including pro-IL-1β, after activation through TLRs. However, inhibition of casp-8 did not affect the production of pro-IL-1β in BMDCs following their interaction with effector CD4⁺ T cells (FIG. 4d). Furthermore, Ripk3^{-/-}Casp8^{-/-} BMDCs had a complete loss of secreted T cell-induced IL-1β (FIG. 4e). Because casp-8 is a mediator of apoptosis induced by cell-extrinsic signals, we investigated if T cell-interacting BMDCs were undergoing cell death. Ripk3^{-/-}Casp8^{-/-} BMDCs were completely resistant to T cell interaction-induced cell death (FIG. 4g). Furthermore, casp-8 dependent BMDC death was mediated by Fas, but not TNFR signaling (FIG. 4h). These observations established a role for casp-8 downstream of Fas signaling in the production of mature IL-1β in T cell-interacting BMDCs.

TNFR-Fas-Dependent IL-1β is Induced in Myeloid Cells

The GM-CSF-derived BMDCs used in this study are a heterogenous mixture of macrophage-like and DC-like cell populations. To examine the importance of the TNFR-Fas pathway in specific cell types of this heterogenous mixture, we sorted CD11c⁺CD11b⁺MHCII^{int} and CD11c⁺CD11b⁺MHC^{hi} BMDCs cells from total BMDCs (FIG. 5g). Both subsets secreted IL-1β in a manner dependent on TNF and FasL after cognate interaction with effector CD4⁺ T cells (FIG. 5a). CD11c⁺ BMDCs are comprised of CSF1R⁺ GM-Macs and CSF1R⁻ GM-DCs. CSF1R⁺ GM-Macs produce IL-1β in response to inflammasome ligands, while CSF1R⁻ GM-DCs do not produce IL-1β or undergo inflammasome activation. To assess whether these subsets differed in their ability to produce inflammasome-independent T cell-induced IL-1β we sorted them based on the expression of CSF1R (FIG. 5h). Purified CSF1R⁺ GM-Macs and CSF1R⁻ GM-DCs were co-cultured with effector CD4⁺ T cells and CD3 Ab. We detected secreted IL-1β in both cultures after 18 h (FIG. 5b). The production of T cell-induced IL-1β in GM-Macs and GM-DCs was blocked when cells were co-cultured with neutralizing antibodies against TNF or FasL (FIG. 5b). Moreover, bone marrow derived macrophages (BMDM) differentiated in the presence of MCSF-containing L929 supernatant for 5 days also produced TNFR-Fas-dependent IL-1β following their interaction with effector CD4⁺ T cells (FIG. 5c). These data suggested that despite the differences between macrophages and DCs to undergo inflammasome activation, both of these myeloid cell populations could use the TNFR-Fas-dependent pathway for the production of IL-1β.

Blocking IL-1R signaling during splenic DC-T cell interaction abrogates the production of effector cytokines by T cells. To test if conventional DCs (cDCs) produced T cell-induced IL-1β, we magnetically sorted CD11c⁺ DCs from the spleens of wild-type mice and co-cultured them with effector CD4⁺ T cells. We detected IL-1β in the culture supernatants after 18 h of CD3 Ab-induced interaction (FIG. 5d). The amount of IL-1β was reduced when cells were cultured with neutralizing antibodies to TNF and FasL (FIG. 5d), indicating that splenic CD11c⁺ cDCs produced T cell-instructed IL-1β in a TNFR-Fas dependent manner. Based on flow cytometry analysis, we could not detect CD11c⁻CSF1R⁺ cells among the splenic CD11c⁺ cells (FIG. 5i), indicating that the production of IL-1β was not due to contamination by macrophages. Zbtb46-GFP mice, in which GFP is expressed concomitantly with the cDC-specific transcription factor ZBTB46, were further used to isolate a pure cDC population and exclude monocyte-derived CD11c⁺GFP⁻Ly6C⁺ DCs. Pure cDCs (CD11c⁺Zbtb46-GFP^{+Ly}6C⁻) (FIG. 5j), were again cultured with effector CD4⁺ T cells and IL-1β was detected in the supernatant following 18 h of culture with CD3 Ab (FIG. 5d). Production of IL-1β was reduced when cells were co-cultured with neutralizing antibodies for TNF or FasL (FIG. 5d).

Because IL-1β can sometimes be critical for the licensing of IL-17A production during effector memory CD4⁺ T cell reactivation, we tested whether TNFR and Fas signaling were necessary for IL-17A production. Compared to wild-type CD11c⁺ cDCs, Tnfrsf1a^{-/-}Tnfrsf1b^{-/-} CD11c⁺ cDCs had diminished capacity to trigger the production of IL-17A in CD62L^{lo}CD44^{hi}CD4⁺ effector memory T cells isolated from the spleen and peripheral LNs, after 18 h of co-culture (FIG. 5e). Blocking FasL using neutralizing antibody during the reactivation of wild-type CD62L^{lo}CD44^{hi}CD4⁺ effector memory T cell by wild-type splenic CD11c⁺ cDCs also reduced the production of IL-17A when compared to PBS control (FIG. 5f). These observations indicated that the TNFR-Fas-dependent mechanism of IL-1β production was conserved in various macrophage and DC populations and enabled optimal CD4⁺ T cell effector function.

T Cell-Induced IL-1β Causes Systemic Inflammation

Although cytokines made by self-reactive T cells contribute to autoimmune inflammation, innate immune activation can precipitate autoimmunity and infiltration of neutrophils and inflammatory monocytes into the affected tissues is a feature of pathology for these diseases. Because the majority of mouse models for T cell-driven autoimmunity rely on initial activation of PRRs, usually through stimulation with Mycobacterium tuberculosis to break tolerance, we used PRR-independent approaches to mimic cognate antigen presenting cell (APC)-T cell interactions that are likely to occur during autoimmune flares. First, we administered CD3 Ab intraperitoneally in wild-type mice to induce systemic TCR activation and widespread reactivation of T cells mediated by myeloid cells. Transcriptional upregulation of Il1b in total splenocytes was detected 4 h after CD3 Ab administration (FIG. 6a). Upregulation of pro-IL-1β protein was detected by flow cytometry in CD11c⁺ cDCs, CD11b⁺Ly6C⁻CSF1R⁺ macrophages, CD11b⁺Ly6G⁻ Ly6C^{int} monocytes and CD11b⁺Ly6C^{hi} inflammatory monocytes isolated from the spleen 3-4 h after administration of CD3 Ab (FIG. 6b and FIG. 6i). No significant induction of pro-IL-1β was observed in spleen CD11b⁺Ly6G⁺ granulocytes (FIG. 6i). Recruitment of Ly6G⁺ neutrophils to the spleen and to the small intestinal lamina propria (SI-LP) was detected within 18 h of CD3 Ab administration (FIG. 6j). Transcriptional upregulation of Il1b or neutrophil infiltration was not detected in Rag1^{-/-} mice injected with CD3 Ab (FIG. 6k,l), indicating that IL-1β induction in this system was dependent on adaptive immune cells. CD3 Ab treatment did not lead to reduction in FoxP3⁺ CD4⁺ T cells in the spleen of wild-type mice (FIG. 6m), indicating the CD3 Ab-induced inflammation was not due to the antibody-mediated depletion of T_{reg} cells. Despite the increased proportion of T_{reg} cells in the spleen (FIG. 6m), CD4⁺ T cells had increased ICOS expression (FIG. 6n), indicating their activated status after CD3 Ab treatment. Next, we administered CD3 Ab intraperitoneally to wild-type or Il1b^{-/-} mice and found less CD11b⁺ monocytes and Ly6G⁺ neutrophils in the spleen and SI-LP of Il1b^{-/-} mice compared to wild-type controls 18 h post-CD3 Ab (FIG. 6c,d and FIG. 6o,p), indicating that systemic inflammation was dependent on production of IL-1β.

In the second approach, OT-II TCR Tg T cells were differentiated in vitro into T_{H}17 cells using polarizing cytokines, adoptively transferred into wild-type recipients and reactivated in vivo with OVA₃₂₃₋₃₃₉ administered intravenously 24 h post-OT-II T_{H}17 transfer. Recruitment of neutrophils to the spleens of wild-type, but not Il1b^{-/-} recipient mice, was observed 12 h post-OVA₃₂₃₋₃₃₉ injection (FIG. 6e). Induction of pro-IL-1β transcripts in splenocytes (FIG. 6f) and accumulation of neutrophils in the spleen (FIG. 6g) was reduced in Tnf^{-/-} mice compared to wild-type controls 4 h post-CD3 Ab administration. Mice with conditional deletion of Fas in CD11c⁺ cells (FIG. 6q) also had reduced neutrophil infiltration in the SI-LP and spleen 18 h post CD3 Ab treatment compared to their Fas^{fl/fl} littermates (FIG. 6h and FIG. 6r). As such, the activation of T cells in vivo resulted in TNF-Fas-dependent production of IL-1β and systemic inflammation.

TNFR-Fas for IL-1β-Mediated Autoimmunity

Self-reactive CD4⁺ T cells are players in several IL-1β-mediated autoimmune diseases, such as multiple sclerosis, RA and type 1 diabetes. IL-1R signaling can sometimes be critical for autoimmune inflammation in EAE. To test if self-reactive CD4⁺ T cells could elicit IL-1β production in EAE, we cultured 2D2 TCR Tg CD4⁺ T cells, which are specific for the myelin oligodendrocyte glycoprotein MOG₃₅₋₅₅ peptide, with wild-type BMDCs in the presence of MOG₃₅₋₅₅ peptide. Production of IL-1β was detected after 18 h in the co-culture supernatant (FIG. 7a), and was reduced when cells were cultured with neutralizing Ab for TNF and FasL (FIG. 7a).

To investigate the mechanism of T cell-induced sterile autoimmune inflammation, we used a passive model of EAE, in which MOG-specific CD4⁺ T cells obtained from MOG-immunized wild-type mice were transferred into naive mice. Prior to transfer into naïve mice, the MOG-specific CD4⁺ T cells were expanded ex vivo and co-cultured with BMDCs to test their ability to induce TNFR-Fas-dependent IL-1β in the BMDCs (FIG. 6s). The MOG-specific CD4⁺ T cells were then transferred intravenously into naïve wild-type or Il1b^{-/-} recipient mice. Unlike the wild-type mice, the Il1b^{-/-} recipient mice were completely resistant to induction of passive EAE by the activated MOG-specific CD4⁺ T cells, as assessed by the induction of progressive paralysis for up to 23 days after transfer (FIG. 7b). Similarly, Tnfrsf1a^{-/-}Tnfrsf1b^{-/-} and lpr recipient mice were protected from MOG-specific CD4⁺ T cell-induced neurological autoimmunity (FIG. 7c,d), and had reduced demyelination of the spinal cord (FIG. 7e) when compared to wild-type recipients. Of note, unmanipulated lpr mice develop a lymphoproliferative disorder as they age, but this does not affect the EAE disease score, which relies on progressive paralysis. After transfer of MOG-specific CD4⁺ T cells, Casp1^{-/-} recipient mice developed disease comparable to wild-type recipients (FIG. 7b), indicating that casp-1 was dispensable for T cell induced auto-immune inflammation. Together, these data provide evidence that autoreactive T cells engaged TNFR and Fas on antigen presenting myeloid cells to induce IL-1β and autoimmune inflammation (FIG. 7f).

Discussion

We showed here that effector CD4⁺ T cells induced IL-1β transcription and cleavage in interacting MPs, including macrophages and DCs, in a TNFR-Fas-dependent manner. We found that effector CD4⁺ T cells of all lineages expressed TNF and FasL that engaged TNFR and Fas on multiple MP subsets, leading to production of IL-1β in these cells in a casp-8-dependent manner. The CD4⁺ T cell-induced IL-1β production was completely independent of PRR activation and did not depend on either canonical or non-canonical inflammasome activation. In a mouse model of passive EAE, T cell-instructed IL-1β appeared to be important for auto-immune pathology. Our studies suggest that the pathway of IL-1β production described here is responsible for inflammation and pathology associated with T cell-driven auto-immune diseases.

While secretion of T cell-induced IL-1β was independent of any PRR activation, its production paralleled that of the inflammasome pathway, where two independent signals appeared to be required for the synthesis and subsequent proteolytic cleavage of pro-IL-1β. The distinction between these two mechanisms of IL-1β production can be further appreciated with regard to their physiological ramifications. The TLR-NLR inflammasome pathway is primarily employed by monocytes and macrophages, but not DCs, to induce IL-1β for clearance of virulent pathogens. In contrast, the T cell-instructed IL-1β, which can be produced by both macrophages and DCs, appears to be responsible for auto-immune inflammation in the absence of overt pathogenic insult. Because DCs do not undergo inflammasome activation, the TNFR-Fas-casp-8 pathway for production of IL-1β appear to be the primary mechanism used by DCs to aid CD4⁺ T cell function, while macrophages appear to employ both pathways for production of IL-1β, depending on the nature of the stimuli.

In addition to TNFR, other TNFR-family members, such as CD40 can also play a role in pro-IL-1β synthesis. The existence of diverse receptors for T cell-induced pro-IL-1β is parallel to the ability of different innate recognition receptors to upregulate pro-IL-1β. Fas signaling has been reported to induce the transcription of pro-IL-1β in TLR9-dependent autoimmune inflammation. We did not find evidence for a role for Fas in the synthesis of pro-IL-1β, suggesting that Fas could contribute to the synthesis of IL-1β in the context of canonical PRR ligands.

Analogous to the inflammasome pathway that culminates in pyroptosis, we observed that MPs underwent cell death upon interaction with T cells. The Fas-casp-8 pathway that appeared to be important for production of T cell-induced IL-1β was also responsible for MP cell death.

Aberrant inflammasome activation caused by gain-of-function mutations in NLRP3 and pyrin drive IL-1β-dependent auto-inflammatory conditions that are also initiated independently of TLRs and other PRRs. However, the role of the inflammasome in T cell-driven autoimmune diseases is neither discernible nor established. While a role for casp-1 in EAE induction has been reported, it is likely due to the ability of Mycobacterium tuberculosis in the MOG emulsion to trigger TLR- and inflammasome-dependent IL-1β. We found that inflammasome was dispensable for T cell-dependent neuroinflammation, although neuroinflammation was dependent on IL-1β.

The ability of T cells to induce IL-1β in MPs engaging the T cells can be a double-edged sword for the health of the host. On one hand, it could license the function of memory CD4⁺ T cells, which can sometimes be critical for anti-microbial immunity, but it could also cause systemic inflammation, as seen in many autoimmune scenarios. It is possible that the TNFR-Fas pathway of IL-1β production evolved primarily to support T cell function during interaction with cDCs. However, the interaction of auto-reactive T cells with macrophages, which might happen in tissues, likely produces higher quantities of IL-1β, which would further contribute to pathology. Because the amount of IL-1β produced by MPs was directly proportional to the concentration of the cognate peptide present, it is unlikely that bystander or low-avidity interaction between MPs and T cells would trigger the production of IL-1β. At the same time, self-reactive CD4⁺ T cells that have escaped thymic selection could engage this pathway during their reactivation, thereby leading to autoimmunity.

Our data provides a mechanism for the role of TNFR and Fas signaling in IL-1β-driven autoimmunity, and suggests that in T cell-mediated autoimmunity, IL-1β is produced through T cell-instruction rather than activation of the inflammasome.

IL-1β is a mediator of anti-microbial immunity as well as autoimmune inflammation. Production of IL-1β can sometimes require transcription by innate immune receptor signaling and maturational cleavage by inflammasomes. In this Example Set, we describe an inflammasome-independent pathway of IL-1β production that was triggered upon cognate interactions between effector CD4⁺ T cells and mononuclear phagocytes (MPs). The cytokine TNF produced by activated CD4⁺ T cells engaged its receptor TNFR on MPs, leading to pro-IL-1β synthesis. Membrane-bound FasL, expressed by CD4⁺ T cells, activated death receptor Fas signaling in MPs resulting in caspase-8-dependent pro-IL-1β cleavage. The T cell-instructed IL-1β resulted in systemic inflammation, while absence of TNFR or Fas signaling protected mice from CD4⁺ T cell-driven autoimmunity. The TNFR-Fas-caspase-8-dependent pathway provides a mechanistic explanation for IL-1β production and its consequences in CD4⁺ T cell-driven autoimmune pathology.

### Example Set 2

The following experiments highlight the role of TNFR signaling and CD40 signaling in inflammation induced by auto-reactive T cells. In this model we deplete regulatory T cells called Treg cells. Depletion of Treg cells leads to activation of self-reactive CD4 T cells and auto-immunity. The understanding in the field has been that auto-reactive T cells attack self, leading to pathology. In this Example Set, we provide evidence that auto-reactive T cells activate the innate immune system by activating TNFR and CD40 on DCs (and macrophages) leading to pathology.

Mice expressing Diptheria Toxin receptor under the control of FOXP3 promoter were used in these experiments. Only Treg cells express DT receptor, hence injection of DT leads to death of these cells leading to activation of auto-reactive T cells and development of auto-immunity. One of the outcomes of Treg depletion is enlargement of spleen and lymph nodes (FIG. 8a). This enlargement is blocked when mice were treated with anti-TNF and ant-CD40L. FIG. 8b shows spleen cell counts from multiple mice. An enlarged spleen (splenomegaly) is sign of inflammation. This is day 5 following DT injection. Antibodies were injected on day minus one and day 3 following DT injection. FIG. 8c shows serum cytokines from the mice on day 5 of DT injection as described above.

FIGS. 8d and 8e show Treg depletion leads to organ damage. Specifically, the lung and liver of mice that have their Treg cells depleted show signs of inflammation including infiltration of neutrophils. This injury as well as neutrophil infiltration is inhibited by blocking TNF and CD40. These data show day 10 after Treg depletion.

### Example Set 3

The following experiments highlight the role of TNFR signaling and CD40 signaling in driving cytokine storm induced by activated T cells. Simultaneous blocking of TNF and CD40 signaling protects mice from cytokine storm induced morbidity and mortality. We demonstrate that activated T cells interact with dendritic cells in vitro to induce IL-6 and IL-12. Similarly, in vivo injection of anti-CD3 antibody (a T cell activator) leads to production of inflammatory cytokines by cells of the innate immune system. Without wishing to be bound by theory, we posit that T cells interact with myeloid cells of the innate immune system through TNF-TNFR and CD40L-CD40 to drive production of IL-6 and 11-12. IL-6 is a driver of pathology during CAR T cell induced cytokine storm as well as cytokine storms seen during viral infections such as COVID-19.

Interaction of effector CD4 T cells with Dendritic cells leads to production of inflammatory cytokines by Dendritic cells. OT-II TCR Tg T cells were differentiated into effector T cells and then were cultured with Bone Marrow DCs in the presence or absence of the OVA peptide. Culture supernatants were collected after 24 hours to measure cytokines by ELISA. DCs produced IL-6 and IL-12 when they were presenting the OVA peptide to effector T cells (FIG. 9a). Similar results were obtained when polyclonal effector T cells were used in the experiment and DC-T cell interaction was induced using anti-CD3 antibody instead of the cognate peptide (FIG. 9b). FIG. 9c shows that inflammatory cytokine production induced by CD4 T cells in DCs is inhibited by blocking TNF and CD40 signaling.

In vivo injection of ant-CD3 (50 μg/mouse) leads to cytokine storm/inflammatory cytokine production (FIG. 9d) that is inhibited by blocking TNF and CD40 (FIG. 9e). Inflammatory cytokines (e.g., IL-6 and IL-12) were measured in the serum, 12 hrs after anti-CD3 injection. Blocking antibodies were administered a two hours before anti-CD3 injection.

FIG. 9f shows that anti-CD3 injection into mice (200 μg/mouse) leads to cytokine storm induced mortality but mice are protected by blocking TNF and CD40 (blockade).

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A method for treating a disease comprising one or more administrations of one or more compositions comprising (a) a TNF signaling inhibitor, (b) a CD40 inhibitor, a FAS signaling inhibitor, or both, and (c) optionally a caspase 8 inhibitor.

2. The method of claim 1, wherein the method for treating the disease comprising one or more administrations of one or more compositions comprising (a) the TNF signaling inhibitor and (b) the CD40 inhibitor.

3. The method of claim 1, wherein the method for treating the disease comprising one or more administrations of one or more compositions comprising (a) the TNF signaling inhibitor, (b) the FAS signaling inhibitor, and (c) optionally the caspase 8 inhibitor.

4. The method of any of the preceding claims, wherein the TNF signaling inhibitor is a TNFR inhibitor, TNFR1 inhibitor, TNFR2 inhibitor, a TNF inhibitor, or a TNFα inhibitor.

5. The method of any of the preceding claims, wherein the TNF signaling inhibitor is a TNFR inhibitor and the TNFR inhibitor is infliximab (Remicade), adalimumab (Humira), certolizumab pegol (Cimzia), golimumab (Simponi), or etanercept (Enbrel).

6. The method of any of the preceding claims, wherein TNF signaling inhibitor is a TNF inhibitor and the TNF inhibitor is infliximab (Remicade) or biosimilars thereof, adalimumab (Humira) or biosimilars thereof, certolizumab or biosimilars thereof, certolizumab pegol (Cimzia) or biosimilars thereof, golimumab (Simponi) or biosimilars thereof, etanercept (Enbrel) or biosimilars thereof, Remsima, Thalidomide (Immunoprin) or derivatives thereof, lenalidomide (Revlimid), pomalidomide (Pomalyst, Imnovid), xanthine derivatives, pentoxifylline, bupropion, 5-HT2A agonist, (R)-DOI, TCB-2, LSD, LA-SS-Az, curcumin, catechins, or Cannabidiol.

7. The method of any of the preceding claims, wherein the CD40 inhibitor is an anti-CD40L antibody, BI 655064, CFZ533, BG9588, or KPL-404.

8. The method of any of the preceding claims, wherein the FAS signaling inhibitor is an anti-FasL Ab.

9. The method of any of the preceding claims, wherein at least one of the one or more compositions comprises the caspase 8 inhibitor and the caspase 8 inhibitor is Emricasan.

10. The method of any of the preceding claims, wherein the amount of the TNFR inhibitor is from about 0.0001% (by weight total composition) to about 99%.

11. The method of any of the preceding claims, wherein the amount of the TNF inhibitor is from about 0.0001% (by weight total composition) to about 99%.

12. The method of any of the preceding claims, wherein the amount of the CD40 inhibitor is from about 0.0001% (by weight total composition) to about 99%.

13. The method of any of the preceding claims, wherein the amount of the FAS inhibitor is from about 0.0001% (by weight total composition) to about 99%.

14. The method of any of the preceding claims, wherein the amount of the caspase 8 inhibitor is from about 0.0001% (by weight total composition) to about 99%.

15. The method of any of the preceding claims, wherein at least one of the one or more compositions further comprises a formulary ingredient.

16. The method of any of the preceding claims, wherein at least one of the one or more compositions is a pharmaceutical composition.

17. The method of any of the preceding claims, wherein at least one of the one or more administrations comprises a parenteral administration, a mucosal administration, an intravenous administration, a depot injection, a subcutaneous administration, a topical administration, an intradermal administration, an oral administration, a sublingual administration, an intratracheal administration, an intranasal administration, an intramuscular administration, an aerosol administration, a nebulizer administration, a pressurized metered-dose inhaler (pMDI) administration, an inhaler administration, or a dry powder inhaler (DPI) administration.

18. The method of any of the preceding claims, wherein at least one of the one or more administrations comprises an intravenous administration, a depot injection, a subcutaneous administration, a topical administration, an oral administration, a sublingual administration, or an intramuscular administration.

19. The method of any of the preceding claims, wherein if there is more than one administration (a) at least one composition used for at least one administration is different from the composition of at least one other administration, (b) the TNF signaling inhibitor is in one composition and the CD40 signaling inhibitor is in another composition, or (c) the TNF signaling inhibitor and the CD40 signaling inhibitor are in same composition.

20. The method of any of the preceding claims, wherein if there is more than one administration (a) at least one composition used for at least one administration is different from the composition of at least one other administration, (b) the TNF signaling inhibitor is in one composition and the FAS signaling inhibitor is in another composition, or (c) the TNF signaling inhibitor and the FAS signaling inhibitor are in same composition.

21. The method of any of the preceding claims, wherein the TNF signaling inhibitor is a TNFR inhibitor and the TNFR inhibitor of at least one of the one or more compositions is administered to the animal in an amount of from about 0.005 mg/kg animal body weight to about 100 mg/kg animal body weight.

22. The method of any of the preceding claims, wherein the TNF signaling inhibitor is a TNF inhibitor and the TNF inhibitor of at least one of the one or more compositions is administered to the animal in an amount of from about 0.005 mg/kg animal body weight to about 100 mg/kg animal body weight.

23. The method of any of the preceding claims, wherein the CD40 inhibitor of at least one of the one or more compositions is administered to the animal in an amount of from about 0.005 mg/kg animal body weight to about 100 mg/kg animal body weight.

24. The method of any of the preceding claims, wherein the FAS inhibitor of at least one of the one or more compositions is administered to the animal in an amount of from about 0.005 mg/kg animal body weight to about 100 mg/kg animal body weight.

25. The method of any of the preceding claims, wherein the caspase 8 inhibitor of at least one of the one or more compositions is administered to the animal in an amount of from about 0.005 mg/kg animal body weight to about 100 mg/kg animal body weight.

26. The method of any of the preceding claims, wherein the animal is a human, a rodent, or a primate.

27. The method of any of the preceding claims, wherein the animal is in need of treatment of the disease.

28. The method of any of the preceding claims, wherein the method is for treating an autoimmune disease, a T cell mediated autoimmune disease, an IL-1β mediated autoimmune disease, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis, uveitis, or cytokine release syndrome.

29. The method of any of the preceding claims, wherein the method is for treating a T cell mediated autoimmune disease, type 1 diabetes, rheumatoid arthritis, multiple sclerosis, or Celiac disease.

30. The method of any of the preceding claims, wherein the method is for treating an IL-1β mediated autoimmune disease, type 1 diabetes, pericarditis, rheumatoid arthritis, or psoriasis.

31. The method of any of the preceding claims, wherein the method is for preventing cytokine release syndrome.

32. A method for treating a disease comprising one or more administrations of one or more compositions comprising (a) a TNF signaling inhibitor and (b) a CD40 inhibitor, wherein the disease is an autoimmune disease, a T cell mediated autoimmune disease, an IL-1β mediated autoimmune disease, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis, uveitis, or cytokine release syndrome.

33. A method for treating a disease comprising one or more administrations of one or more compositions comprising (a) a TNF signaling inhibitor, (b) a FAS signaling inhibitor, and (c) optionally a caspase 8 inhibitor, wherein the disease is an autoimmune disease, a T cell mediated autoimmune disease, an IL-1β mediated autoimmune disease, transplant rejection, type 1 diabetes, rheumatoid arthritis, psoriatic arthritis, ankylosing spondylitis, juvenile rheumatoid arthritis, multiple sclerosis, graft vs. host disease, Celiac disease, inflammatory bowel disease, pericarditis, psoriasis, Crohn's disease, ulcerative colitis, uveitis, or cytokine release syndrome.
